(19) United States
(12) Patent Application Publication
(10) Pub. No.: US 2012/0220595 A1
(43) Pub. Date: Aug. 30, 2012

(54) Deuterated Tyrosine Kinase Inhibitors

(75) Inventors: David M. Epstein, Huntington, NY (US); Meizhong Jin, Dix Hills, NY (US); Mark J. Mulvihill, Dix Hills, NY (US)

(73) Assignee: OSI Pharmaceuticals, LLC

(21) Appl. No.: 13/504,979

(22) PCT Filed: Nov. 11, 2010
(86) PCT No.: PCT/US10/56290

§ 371 Date: Apr. 30, 2012

Related U.S. Application Data
(60) Provisional application No. 61/260,447, filed on Nov. 12, 2009.

(51) Int. Cl. A61K 31/53; A61K 31/4985; A61P 35/00; C07D 487/04

(52) U.S. Cl. 514/243; 544/350; 514/249; 544/184

(57) ABSTRACT

Compounds of Formula I, as shown below and defined herein: enriched in deuterium, and pharmaceutically acceptable salts thereof, synthesis, intermediates, formulations, and methods of disease treatment therewith, including cancers mediated at least in part by IGF-1R and/or IR.

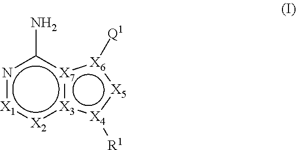

This application claims all benefits of priority U.S. Appl. No. 61/260,447 (Nov. 12, 2009), which is incorporated herein in its entirety by this reference.

### FIELD AND BACKGROUND

The present invention pertains at least in part to cancer treatment, certain chemical compounds, and methods of treating tumors and cancers with the compounds.

The development of target-based anti-cancer therapies has become the focus of a large number of pharmaceutical research and development programs. Various strategies of intervention include targeting protein tyrosine kinases, including receptor tyrosine kinases believed to drive or mediate tumor growth.

Insulin-like growth factor-1 receptor (IGF-1R) is a receptor tyrosine kinase that plays a key role in tumor cell proliferation and apoptosis inhibition, and has become an attractive cancer therapy target. IGF-1R is involved in the establishment and maintenance of cellular transformation, is frequently overexpressed by human tumors, and activation or overexpression thereof mediates aspects of the malignant phenotype. IGF-1R activation increases invasion and metastasis propensity.

Inhibition of receptor activation has been an attractive method having the potential to block IGF-mediated signal transduction. Anti-IGF-1R antibodies to block the extracellular ligand-binding portion of the receptor and small molecules to target the enzyme activity of the tyrosine kinase domain have been developed.

See, e.g., Expert Opin. Ther. Patents, 17(1):25-35 (2007);Expert Opin. Ther. Targets, 12(5):589-603 (2008); Am J. Transi. Res., 1:101-114 (2009).

US 2006/0235031 (published Oct. 19, 2006) describes a class of bicyclic ring substituted protein kinase inhibitors, including Example 31 thereof, which corresponds to the IGF-1R inhibitor known as OSI-906. OSI-906 is in clinical development in various tumor types.

Pharmaceutically active compounds enriched in deuterium at designated positions have been proposed. These have included tyrosine kinase inhibitors and other compounds, such as in US 2009/0185999 and US 2009/0209592. See also Sci. & Tech., 87(25), pp. 36-39 (Jun. 22, 2009).

There is a continuing need for effective therapies for use in proliferative disease, including treatments for primary cancers, prevention of metastatic disease, and targeted therapies, including tyrosine kinase inhibitors, such as IGF-1R and/or IR inhibitors, dual inhibitors, including selective inhibitors, and for potent, orally bioavailable, and efficacious inhibitors.

### SUMMARY

In some aspects, the present invention concerns compounds of Formula I, as shown below and defined herein, which are enriched in deuterium (D) at any one or more selected positions.

In some aspects, the present invention provides a compound of Formula I:

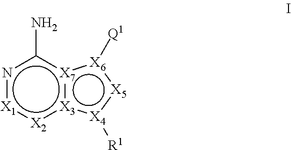

or a pharmaceutically acceptable salt thereof, wherein:

X₁ and X₂ are each independently N or >CE¹; X₃, X₄, X₆, and X₇ are each independently N or C; X₅ is N, >CH, >CD, or >NE¹; wherein at least one of X₃, X₄, X₅, X₆, and X₇ is N or >NE¹;

Q¹ is

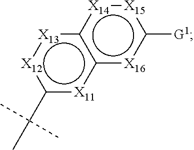

wherein X₁₁, X₁₂, X₁₃, X₁₄, X₁₅, and X₁₆ are each independently N, >C-E¹¹, or >N⁺—O⁻; and at least one of X₁₁, X₁₂, X₁₃, X₁₄, X₁₅, and X₁₆ is N or >N⁺—O⁻;

and R¹, G¹, and each E¹¹ represent optional substituents that may incorporate deuterium; and wherein

at least one hydrogen atom of the compound is enriched in deuterium (D).

The invention includes the compounds and pharmaceutically acceptable salts thereof.

In some aspects, compounds of the invention are inhibitors of kinases, including at least one of IGF-1R and IR.

In some aspects, compounds of the invention are selective inhibitors of IGF-1R and IR.

In some aspects, the invention includes treating proliferative disease, particularly cancers, including cancers mediated by IGF-1R and/or IR, alone or in combination regimens with other agents.

The invention includes the compounds and salts thereof, and their physical forms, preparation of the compounds, useful intermediates, and pharmaceutical compositions and formulations thereof.

### DETAILED DESCRIPTION

### Compounds

In some aspects, the present invention concerns compounds and salts thereof of Formula I, as shown below and defined herein:

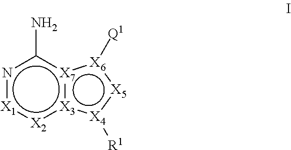

wherein X₁, and X₂ are each independently N or CE¹

X₃, X₄, X₆, and X₇ are each independently N or C;

X₅ is N, >CH, >CD, or >NE¹;

wherein at least one of X₃, X₄, X₅, X₆, and X₇ is N or >NE¹;

Q¹ is

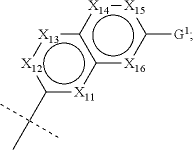

wherein X₁₁, X₁₂, X₁₃, X₁₄, X₁₅, and X₁₆ are each independently N, >C-E¹¹, or >N⁺—O⁻; and at least one of X₁₁, X₁₂, X₁₃, X₁₄, X₁₅, and X₁₆ is N or >N⁺—O⁻;

E¹ is H, D, halo, —CF₃, —OCF₃, —OR², —NR²R³(R^{2a})ₒ, —C(═O)R², —CO₂R², —CONR²R³, —NO₂, —CN, —S(O)ⱼ₁R², —SO₂NR²R³, —NR²C(═O)R³, —NR²C(═O)OR³, —NR²C(═O)NR³R^{2a}, —NR²S(O)ⱼ₁R³, —C(═S)OR², —C(═O)SR², —NR²C(═NR³)NR^{2a}R^{3a}, —NR²C(═NR³)OR^{2a}, —NR²C(═NR³)SR^{2a}, —OC(═O)OR², —C(═O)NR²R³, —OC(═O)SR², —SC(═O)OR², —SC(═O)NR²R³, C₀₋₁₀alkyl, C₂₋₁₀alkenyl, C₂₋₁₀alkynyl, C₁₋₁₀alkoxyC₂₋₁₀alkenyl, C₁₋₁₀alkoxyC₂₋₁₀alkynyl, C₁₋₁₀alkylthioC₁₋₁₀alkyl, C₁₋₁₀alkylthioC₂₋₁₀alkenyl, C₁₋₁₀alkylthioC₂₋₁₀alkynyl, cycloC₃₋₈alkyl, cycloC₃₋₈alkenyl, cycloC₃₋₈alkylC₁₋₁₀alkyl, cycloC₃₋₈alkenylC₁₋₁₀alkyl, C₃₋₈alkylC₂₋₁₀alkenyl, cycloC₃₋₈alkenylC₂₋₁₀alkenyl, cycloC₃₋₈alkylC₂₋₁₀alkynyl, cycloC₃₋₈alkenylC₂₋₁₀alkynyl, heterocyclyl-C₀₋₁₀alkyl, heterocyclyl-C₂₋₁₀alkenyl, or heterocyclyl-C₂₋₁₀alkynyl, any of which is optionally substituted with one or more independent D, halo, oxo, —CF₃, —OCF₃, —OR²²², —NR²²²R³³³(R^{222a})ⱼ₁ₐ, —C(═O)R²²², —CO₂R²²², —C(═O)NR²²²R³³³, —NO₂, —CN, —S(═O)SO₂NR²²²R³³³, —NR²²²C(═O)R³³³, —NR²²²C(═O)OR³³³, —NR²²²C(═O)NR³³³R^{222a}, —NR²²²S(O)ⱼ₁ₐR³³³, —C(═S)OR²²², —C(═O)SR²²², —NR²²²C(═NR³³³)NR^{222a}R^{333a}, —NR²²²C(═NR³³³)OR^{222a}, —NR²²²C(═NR³³³)SR^{222a}, —C(═O)OR²²², —OC(═O)NR²²²R³³³, —C(═O)SR²²², —SC(═O)OR²²², or —SC(═O)NR²²²R³³³ substituents;

or E¹ is aryl-C₀₋₁₀alkyl, aryl-C₂₋₁₀alkenyl, aryl-C₂₋₁₀alkynyl, hetaryl-C₀₋₁₀alkyl, hetaryl-C₂₋₁₀alkenyl, or hetaryl-C₂₋₁₀alkynyl, any of which is optionally substituted with one or more independent D, halo, —CF₃, —OCF₃, —OR²²², —NR²²²R³³³(R^{222a})ⱼ₂ₐ, —C(O)R²²², —CO₂R²²², —C(═O)NR²²²R³³³, —NO₂, —CN, —S(O)_{J2a}R²²², —SO₂NR²²²R³³³, —NR²²²C(═O)R³³³, —NR²²²C(═O)OR³³³, —NR²²²C(═O)NR³³³R^{222a}, —NR²²²S(O)ⱼ₂ₐR³³³, —O(═S)OR²²², —C(═O)SR²²², —NR²²²C(═NR³³³)NR^{222a}R^{333a}, —NR²²²C(═NR³³³)OR^{222a}, —NR²²²C(═NR³³³)SR^{222a}, —OC(═O)OR²²², —OC(═O)NR²²²R³³³, —OC(═O)SR²²², —SC(═O)OR²²², or —SC(═O)NR²²²R³³³ substituents;

each E¹¹ is independently H, D, halo, —CF₃, —OCF₃, methyl, or ethyl;

G¹ is phenyl or pyridyl, either optionally substituted by one or more D or halogen atoms;

R¹ is absent, D, C₀₋₁₀alkyl, cycloC₃₋₁₀alkyl, bicycloC₅₋₁₀alkyl, aryl, heteroaryl, aralkyl, heteroaralkyl, heterocyclyl, heterobicycloC₅₋₁₀alkyl, spiroalkyl, or heterospiroalkyl, any of which is optionally substituted by one or more independent G¹¹ substituents;

each G¹¹ is H, D, halo, oxo, —CF₃, —OCF₃, —OR²¹, —NR²¹R³¹(R^{2a1})ⱼ₄, —C(O)R²¹, —CO₂R²¹, —C(═O)NR²¹R³¹, —NO₂, —CN, —S(O)ⱼ₄R²¹, —SO₂NR²¹R³¹, NR²¹(C═O)R³¹, NR²¹C(═O)OR³¹, NR²¹C(═O)NR³¹R^{2a1}, NR²¹S(O)ⱼ₄R³¹, —C(═S)OR²¹, —C(═O)SR²¹, —NR²¹C(═NR³¹)NR^{2a1}R^{3a1}, NR²¹C(═NR³¹)OR^{2a1}, —NR²¹C(═NR³¹)SR^{2a1}, —OC(═O)OR²¹, —OC(═O)NR²¹R³¹, —OC(═O)SR²¹, —SC(═O)OR²¹, —SC(═O)NR²¹R³¹, —P(O)OR²¹OR³¹, C₁₋₁₀alkylidene, C₀₋₁₀alkyl, C₂₋₁₀alkenyl, C₂₋₁₀alkynyl, C₁₋₁₀alkoxyC₁₋₁₀alkyl, C₁₋₁₀alkoxyC₂₋₁₀alkenyl, C₁₋₁₀alkoxyC₂₋₁₀alkynyl, C₁₋₁₀alkylthioC₁₋₁₀alkyl, C₁₋₁₀alkylthioC₂₋₁₀alkenyl, C₁₋₁₀alkylthioC₁₋₁₀alkynyl, cycloC₃₋₈alkyl, cycloC₃₋₈alkenyl, cycloC₃₋₈alkylC₁₋₁₀alkyl, cycloC₃₋₈alkenylC₁₋₁₀alkyl, cycloC₃₋₈alkylC₂₋₁₀alkenyl, cycloC₃₋₈alkenylC₂₋₁₀alkenyl, cycloC₃₋₈alkylC₂₋₁₀alkynyl, cycloC₃₋₈alkenylC₂₋₁₀alkynyl, heterocyclyl-C₀₋₁₀alkyl, heterocyclyl-C₂₋₁₀alkenyl, or heterocyclyl-C₂₋₁₀alkynyl,

any of which is optionally substituted with one or more independent D, halo, oxo, —CF₃, —OCF₃, —OR²²²¹, —NR²²²¹R³³³¹(R^{222a1}) C(O)R²²²¹, —CO₂R²²²¹, —C(═O)NR²²²¹R³³³¹, —NO₂, —CN, —S(O)ⱼ₄ₐR²²²¹, —SO₂NR²²²¹R³³³¹, —NR²²²¹C(═O)R³³³¹, —NR²²²¹C(═O)OR³³³¹, —NR²²²¹C(═O)NR³³³¹R^{222a1}, —NR²²²¹S(O)ⱼ₄ₐR³³³¹, —C(═S)OR²²²¹, —C(═O)SR²²²¹, —NR²²²¹C(═NR³³³¹)NR^{222a1}R^{333a1}, —NR²²²¹C(═NR³³³¹)OR^{222a1}, —NR²²²¹C(═NR³³³¹)SR^{222a1}, —OC(═O)OR²²²¹, —OC(═O)NR²²²¹R³³³¹, —OC(═O)SR²²²¹, —SC(═O)OR²²²¹, —P(O)OR²²²¹OR³³³¹, or —SC(═O)NR²²²¹R³³³¹ substituents;

or G¹¹ is aryl-C₀₋₁₀alkyl, aryl-C₂₋₁₀alkenyl, aryl-C₂₋₁₀alkynyl, hetaryl-C₀₋₁₀alkyl, hetaryl-C₂₋₁₀alkenyl, or hetaryl-C₂₋₁₀alkynyl, any of which is optionally substituted with one or more independent D, halo, —CF₃, —OCF₃, —OR²²²¹, —NR²²²¹R³³³¹(R^{222a1})ⱼ₅ₐ, —C(O)R²²²¹, —CO₂R²²²¹, —C(═O)NR²²²¹R³³³¹, —NO₂, —CN, —S(O)ⱼ₅ₐR²²²¹, —SO₂NR²²²¹R³³³¹, NR²²²¹C(═O)R³³³¹, —NR²²²¹, —NR²²²¹C(═O)OR³³³¹, —NR²²²¹C(═O)NR³³³¹R^{222a1}, —NR²²²¹S(O)ⱼ₅ₐR³³³¹, —C(═S)OR²²²¹, —C(═O)SR²²²¹, —NR²²²¹C(═NR³³³¹)NR^{222a1}R^{333a1}, —NR²²²¹C(═NR³³³¹)OR^{222a1}, —NR²²²¹C(═NR³³³¹)SR^{222a1}, —OC(═O)OR²²²¹, —OC(═O)NR²²²¹R³³³¹, —OC(═O)SR²²²¹, —SC(═O)OR²²²¹, —P(O)OR²²²¹OR³³³¹, or —SC(═O)NR²²²¹R³³³¹ substituents;

or G¹¹ is C, taken together with the carbon to which it is attached forms a C═C double bond which is substituted with R⁵ and G¹¹¹;

R², R^{2a}, R³, R^{3a}, R²²², R^{222a}, R³³³, R^{333a}, R²¹, R^{2a1}, R³¹, R^{3a1}, R²²²¹, R^{222a1}, R³³³¹, and R^{333a1} are each independently C₀₋₁₀alkyl, C₂₋₁₀alkenyl, C₂₋₁₀alkynyl, C₁₋₁₀alkoxyC₁₋₁₀alkyl, C₁₋₁₀alkoxyC₂₋₁₀alkenyl, C₁₋₁₀alkoxyC₂₋₁₀alkynyl, C₁₋₁₀alkylthioC₂₋₁₀alkenyl, C₁₋₁₀alkylthioC₂₋₁₀alkynyl, cycloC₃₋₈alkyl, cycloC₃₋₈alkenyl, cycloC₃₋₈alkylC₁₋₁₀alkyl, cycloC₃₋₈alkenylC₁₋₁₀alkyl, cycloC₃₋₈alkylC₂₋₁₀alkenyl, cycloC₃₋₈alkenylC₂₋₁₀alkenyl, cycloC₃₋₈alkylC₂₋₁₀alkynyl, cycloC₃₋₈alkenylC₂₋₁₀alkynyl, heterocyclyl-C₀₋₁₀alkyl, heterocyclyl-C₂₋₁₀alkenyl, heterocyclyl-C₂₋₁₀alkynyl, aryl-C₀₋₁₀alkenyl, or aryl-C₂₋₁₀alkynyl, hetaryl-C₀₋₁₀alkyl, hetaryl-C₂₋₁₀alkenyl, or hetaryl-C₂₋₁₀alkynyl, any of which is optionally substituted by one or more independent G¹¹¹ substituents;

or in the case of —NR²R³(R^{2a})ⱼ₁ or —NR²²²R³³³³(R^{222a})ⱼ₁ₐ or —NR²²²R³³³(R^{222a})ⱼ₂ₐ or —NR²¹R³¹(R^{2a1})ⱼ₄, —NR²²²¹R³³³¹(R^{222a1})ⱼ₄ or —NR²²²¹R³³³¹(R^{222a1})ⱼ₅ₐ then R² and R³, or R²²² and R³³³, or R²²²¹ and R³³³¹, respectively, are optionally taken together with the nitrogen atom to which they are attached to form a 3-10 membered saturated or unsaturated ring, wherein said ring is optionally substituted by one or more independent G¹¹¹¹ substituents and wherein said ring optionally includes one or more heteroatoms other than the nitrogen to which R² and R³, or R²²² and R³³³, or R²²²¹ and R³³³¹ are attached;

R⁵, G¹¹¹, and G¹¹¹¹ are each independently C₀₋₁₀alkyl, C₂₋₁₀alkenyl, C₂₋₁₀alkynyl, C₁₋₁₀alkoxyC₁₋₁₀alkyl, C₁₋₁₀alkoxyC₂₋₁₀alkenyl, C₁₋₁₀alkoxyC₂₋₁₀alkynyl, C₁₋₁₀alkylthioC₂₋₁₀alkenyl, C₁₋₁₀alkylthioC₂₋₁₀alkynyl, cycloC₃₋₈alkyl, cycloC₃₋₈alkenyl, cycloC₃₋₈alkylC₁₋₁₀ alkyl, cycloC₃₋₈alkenylC₁₋₁₀alkyl, cycloC₃₋₈alkylC₂₋₁₀alkenyl, cycloC₃₋₈alkenylC₂₋₁₀alkenyl, cycloC₃₋₈alkylC₂₋₁₀alkynyl, cycloC₃₋₈alkenylC₂₋₁₀alkynyl, heterocyclyl-C₀₋₁₀alkyl, heterocyclyl-C₂₋₁₀alkenyl, heterocyclyl-C₂₋₁₀alkynyl, aryl-C₂₋₁₀alkenyl, aryl-C₂₋₁₀alkynyl, hetaryl-C₀₋₁₀alkyl, hetaryl-C₂₋₁₀alkenyl, or hetaryl-C₂₋₁₀alkynyl, any of which is optionally substituted with one or more independent D, halo, —CF₃, —OCF₃, —OR⁷⁷, —NR⁷⁷R⁸⁷, —C(O)R⁷⁷, —CO₂R⁷⁷, —CONR⁷⁷R⁸⁷, —NO₂, —CN, —S(O)ⱼ₅ₐR⁷⁷, —SO₂NR⁷⁷R⁸⁷, —NR⁷⁷C(═O)R⁸⁷, —NR⁷⁷C(═O)OR⁸⁷, —NR⁷⁷C(═O)NR⁷⁸R⁸⁷, —NR⁷⁷S(O)_{J5a}R⁸⁷, —C(═S)OR⁷⁷, —C(═O)SR⁷⁷, —NR⁷⁷C(═NR⁸⁷)NR⁷⁸R⁸⁸, —NR⁷⁷C(═NR⁸⁷)OR⁷⁸, —NR⁷⁷C(═NR⁸⁷)SR⁷⁸, —OC(═O)OR⁷⁷, —OC(═O)NR⁷⁷R⁸⁷, —OC(═O)SR⁷⁷, —SC(═O)OR⁷⁷, —P(O)OR⁷⁷OR⁸⁷, or —SC(═O)NR⁷⁷R⁸⁷ substituents;

R⁷⁷, R⁷⁸, R⁸⁷, and R⁸⁸ are each independently C₀₋₁₀alkyl, C₂₋₁₀alkenyl, C₂₋₁₀alkynyl, C₁₋₁₀alkoxyC₁₋₁₀alkyl, C₁₋₁₀alkoxyC₂₋₁₀alkenyl, C₁₋₁₀alkoxyC₂₋₁₀alkynyl, C₁₋₁₀alkylthioC₁₋₁₀alkyl, C₁₋₁₀alkylthioC₂₋₁₀alkenyl, C₁₋₁₀alkylthioC₂₋₁₀alkynyl, cycloC₃₋₈alkyl, cycloC₃₋₈alkenyl, cycloC₃₋₈alkylC₁₋₁₀alkyl, cycloC₃₋₈alkenylC₁₋₁₀alkyl, cycloC₃₋₈alkylC₂₋₁₀alkenyl, cycloC₃₋₈alkenylC₂₋₁₀alkenyl, cycloC₃₋₈alkylC₂₋₁₀alkynyl, cycloC₃₋₈alkenylC₂₋₁₀alkynyl, heterocyclyl-C₀₋₁₀alkyl, heterocyclyl-C₂₋₁₀alkenyl, heterocyclyl-C₂₋₁₀alkynyl, C₁₋₁₀alkylcarbonyl, C₂₋₁₀alkenylcarbonyl, C₂₋₁₀alkynylcarbonyl, C₁₋₁₀alkoxycarbonyl, C₁₋₁₀alkoxycarbonylC₁₋₁₀alkyl, monoC₁₋₆alkylaminocarbonyl, diC₁₋₆alkylaminocarbonyl, mono(aryl)aminocarbonyl, di(aryl)aminocarbonyl, or C₁₋₁₀alkyl(aryl)aminocarbonyl, any of which is optionally substituted with one or more independent halo, cyano, hydroxy, nitro, C₁₋₁₀alkoxy, —SO₂N(C₀₋₄alkyl)(C₀₋₄alkyl), or —N(C₀₋₄alkyl)(C₀₋₄alkyl) substituents;

or R⁷⁷, R⁷⁸, R⁸⁷, and R⁸⁸ are each independently aryl-C₀₋₁₀alkyl, aryl-C₂₋₁₀alkenyl, aryl-C₂₋₁₀alkynyl, hetaryl-C₀₋₁₀alkyl, hetaryl-C₂₋₁₀alkenyl, hetaryl-C₂₋₁₀alkynyl, mono(C₁₋₆alkyl)aminoC₁₋₆alkyl, di(C₁₋₆alkyl)aminoC₁₋₆alkyl, mono(aryl)aminoC₁₋₆alkyl, di(aryl)aminoC₁₋₆alkyl, or —N(C₁₋₆alkyl)-C₁₋₆alkyl-aryl, any of which is optionally substituted with one or more independent halo, cyano, nitro, —O(C₀₋₄alkyl), C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CON(C₀₋₄alkyl)(C₀₋₁₀alkyl), —SO₂N(C₀₋₄alkyl)(C₀₋₄alkyl), or —N(C₀₋₄alkyl)(C₀₋₄alkyl) substituents;

j1, j1a, j2a, j4, j4a, and j5a are each independently 0, 1, or 2;

or a pharmaceutically acceptable salt thereof, wherein any hydrogen atom can be replaced by a D atom and the compound or salt is present as a material comprising at least one D atom in an abundance of at least about 10%.

In some aspects of the invention, the compound or salt described above can have the formula:

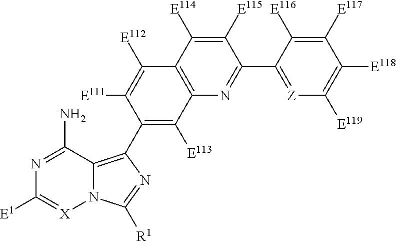

wherein:

X is N, >CH, or >CD;

Z is N, >CH, >CD, or >C-halogen;

R¹ is phenyl, cycloC₃₋₆alkyl, bicycloC₅₋₁₀alkyl, spiroalkyl, or heteroalkyl, any of which is optionally substituted by one or more G¹¹ substituents;

E¹ is H or D;

each E¹¹¹-E¹¹⁵ is independently H, D, halogen, —CF₃, methyl, or ethyl;

each E¹¹⁶-E¹¹⁹ is independently H, D, or halogen;

which is present as a material comprising at least one D atom in an abundance of at least about 20%.

In some aspects of the inventions described above, R¹ is cycloC₃₋₆alkyl optionally substituted by one or more G¹¹ substituents.

In some aspects of the inventions described above, the compound or salt has the formula:

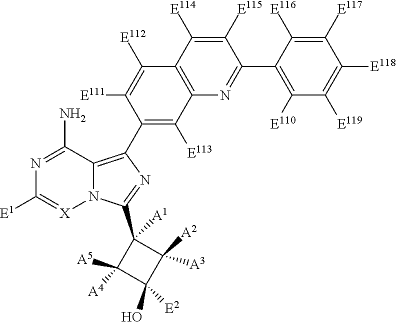

wherein:

X is N, >CH, or >CD;

E¹ is H or D;

each E¹¹¹-E¹¹⁵ is independently H, D, halo, —CF₃, or methyl;

each E¹¹⁰ and E¹¹⁶-E¹¹⁹ is independently H, D, or halogen;

each A¹-A⁵ is independently H or D,

E² is —CH₃, CH₂D, CHD₂, or CD₃; and

which is present as a material comprising at least one said D atom in an abundance of at least about 30%.

In some aspects of the inventions described above,

X is CH or CD;

each of A¹-A⁵, E¹, E¹¹⁰-E¹¹⁹ is independently H or D;

E² is —CH₃, CH₂D, CHD₂, or CD₃; and

which is present as a material comprising at least one said D atom in an abundance of at least about 40%.

In some aspects of the invention, the compound is present as a material comprising at least 1 to 3 said D atoms each in an abundance of at least about 50%. In some aspects of the invention, the compound is present as a material comprising the incorporated D atom(s) each in an abundance of at least about 50%. In some aspects of the invention, the compound is present as a material in which each atom designated as deuterium has an deuterium incorporation of at least about 50%. However, according to the present invention, the abundance of a deuterium atom at a selected position can be at least about 10%, 30%, 50%, 70%, 90%, or greater.

In some aspects of the invention, the compound is present as a material in which at least one atom designated as deuterium has an deuterium incorporation of at least about 70%.

In some aspects of the invention, the compound is present as a material in which each atom not designated as deuterium has substantially its natural isotopic abundance.

In some aspects of the invention, the compound inhibits IGF-1R with an IC₅₀ of about 1 μM or less in a cellular assay.

In some aspects of the invention, the compound is present as a material that is substantially stereochemically pure.

In some aspects of the invention, the compound is present in pharmaceutical composition comprising the compound or salt formulated with or without one or more pharmaceutical carriers.

In some aspects of the invention, there is provided a method of treating cancer mediated at least in part by IGF-1R comprising administering to a mammal in need thereof a therapeutically effective amount of a compound or salt or composition of the invention.

In some aspects of the invention, there is provided a method of treating cancer selected from non-small cell lung, adrenocortical carcinoma, colorectal, ovarian, multiple myeloma, pancreatic, squamous call head and neck, prostate, sarcoma, anaplastic thyroid, renal cell carcinoma, small cell lung, or gastric cancer, comprising administering to a mammal in need thereof a therapeutically effective amount of a compound or salt of the invention in a single agent regimen or with the administration of one or more additional anti-cancer agents.

In some aspects of the invention, such a method comprises administering a therapeutically effective amount of at least one additional anti-cancer agent.

In some aspects of the invention, the additional anti-cancer agent(s) comprises a chemotherapy agent.

In some aspects of the invention, the additional anti-cancer agent(s) comprises a molecular targeted therapy agent.

In some aspects of the invention, such a method comprises administering a therapeutically effective amount of at least one EGFR inhibitor.

In some aspects of the invention, the cancer comprises non-small cell lung cancer.

Each variable definition above includes any subset thereof and the compounds of Formula I include any combination of such variables or variable subsets.

In some aspects, the invention includes any of the compound examples herein and pharmaceutically acceptable salts thereof.

The invention includes the compounds and salts thereof, and their physical forms, preparation of the compounds, useful intermediates, and pharmaceutical compositions and formulations thereof.

Compounds described can contain one or more asymmetric centers and may thus give rise to stereoisomers. The present invention includes any stereoisomers, even if not specifically shown, individually as well as mixtures, geometric isomers, and pharmaceutically acceptable salts thereof. Where a compound or stereocenter is described or shown without definitive stereochemistry, it is to be taken to embrace all possible individual isomers, configurations, and mixtures thereof. Thus, a material sample containing a mixture of stereoisomers would be embraced by a recitation of either of the stereoisomers or a recitation without definitive stereochemistry. Also contemplated are any cis/trans isomers or tautomers of the compounds described.

Further, the compounds may be amorphous or may exist or be prepared in various crystal forms or polymorphs, including solvates and hydrates. The invention includes any such forms provided herein, at any purity level. A recitation of a compound per se means the compound regardless of any unspecified stereochemistry, physical form and whether or not associated with solvent or water. A recitation of a compound also includes any isotopes thereof.

When a tautomer of the compound of Formula (I) exists, the compound of formula (I) of the present invention includes any possible tautomers and pharmaceutically acceptable salts thereof, and mixtures thereof, except where specifically stated otherwise.

Compounds of the invention may be referred to as isotopologs in that they differ from their corresponding non-enriched compounds only in the isotopic composition thereof.

Alternatively or in addition, isotopes other than D may be incorporated into the compounds.

### Preparation

The invention includes the intermediates, examples, and synthetic methods described herein.

The compounds of the Formula I may be prepared by the methods described below, together with synthetic methods known in the art of organic chemistry, or modifications and derivatizations that are familiar to those of ordinary skill in the art. The starting materials used herein are commercially available or may be prepared by routine methods known in the art (such as those methods disclosed in standard reference books such as the COMPENDIUM OF ORGANIC SYNTHETIC METHODS, Vol. I-VI (published by Wiley-Interscience)). Preferred methods include those described below.

During any of the following synthetic sequences it may be necessary and/or desirable to protect sensitive or reactive groups on any of the molecules concerned. This can be achieved by means of conventional protecting groups, such as those described in T. W. Greene, Protective Groups in Organic Chemistry, John Wiley & Sons, 1981; T. W. Greene and P. G. M. Wuts, Protective Groups in Organic Chemistry, John Wiley & Sons, 1991, and T. W. Greene and P. G. M. Wuts, Protective Groups in Organic Chemistry, John Wiley & Sons, 1999, which are hereby incorporated by reference.

Compounds of Formula I, or their pharmaceutically acceptable salts, can be prepared according to the reaction Schemes discussed hereinbelow and the general skill in the art. Unless otherwise indicated, the substituents in the Schemes are defined as above. Isolation and purification of the products is accomplished by standard procedures, which are known to a chemist of ordinary skill.

### General Synthetic Methods

Compounds of the invention may be prepared by the methods described below, together with synthetic methods known in the art of organic chemistry, or modifications and derivatizations that are familiar to those of ordinary skill in the art. See, e.g., US2006/0084654; US2006/0235031; US2007/0129547; and US2007/0149521, which are incorporated herein in their entireties for all purposes, including synthetic methods.

Reference is made to the above-noted publications, in particular US2006/0235031, Schemes 1-11 for general methods of preparing non-deuterated analogs of the compounds of the present invention. Below are further described methods of incorporating deuterium in the compounds of the invention, which can be used in conjunction with known methods.

Schemes 1-3 generally describe incorporation of deuterium in an imidazo[1,5-a]pyrazine core, followed by halogenation, ammonolysis and installation of Q¹ group.

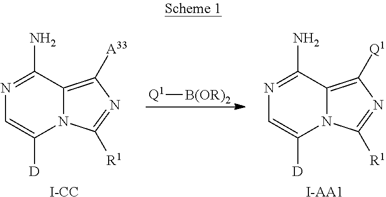

Q¹ and R¹ are as defined previously for compound of Formula I, A³³=halogen such as Cl, Br, or I and B(OR)₂=suitable boronic acid/esters.

In a typical preparation of compounds of Formula I-AA1, compound of Formula I-CC may be reacted with a suitable boronic acid/ester (Q¹-B(OR)₂) in a suitable solvent via typical Suzuki coupling procedures. Suitable solvents for use in the above process may include, but are not limited to, ethers such as THF, glyme, dioxane, dimethoxyethane, and the like; DMF; DMSO; MeCN; alcohols such as MeOH, EtOH, isopropanol, trifluoroethanol, and the like; and chlorinated solvents such as DCM or chloroform (CHCl₃). If desired, mixtures of these solvents may be used, however, the preferred solvent may be dimethoxyethane/water. The above process may be carried out at temperatures between about −78° C. and about 120° C. Preferably, the reaction may be carried out between 60° C. and about 100° C. The above process to produce compounds of the present invention may be preferably carried out at about atmospheric pressure although higher or lower pressures may be used if desired. Substantially, equimolar amounts of reactants may be preferably used although higher or lower amounts may be used.

One skilled in the art will appreciate that alternative methods may be applicable for preparing compounds of Formula I-AA1 from I-CC. For example, compound of Formula I-CC may be reacted with a suitable organotin reagent Q¹-SnBu₃ or the like in a suitable solvent via typical Stille coupling procedures.

The compounds of Formula I-CC of Scheme 1 may be prepared as shown below in Scheme 2.

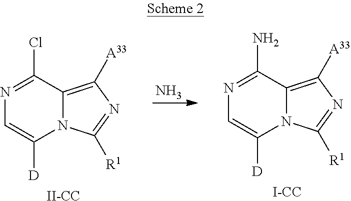

R¹ is as defined previously for compound of Formula I and A³³=halogen such as Cl, Br, or I.

In a typical preparation of compounds of Formula I-CC, compound of Formula II-CC may be reacted with ammonia in a suitable solvent. Suitable solvents for use in the above process may include, but are not limited to, ethers such as THF, glyme, and the like; DMF; DMSO; MeCN; alcohols such as MeOH, EtOH, isopropanol, trifluoroethanol, and the like; and chlorinated solvents such as DCM or chloroform (CHCl₃). If desired, mixtures of these solvents may be used, however, the preferred solvents may be isopropanol and a mixture of THF and isopropanol. The above process may be carried out at temperatures between about −78° C. and about 120° C. Preferably, the reaction may be carried out between 80° C. and about 120° C. The above process to produce compounds of the present invention may be preferably carried in a sealed reaction vessel such as but not limited to a thick walled glass reaction vessel or a stainless steel Parr bomb. An excess amount of the reactant, ammonia, may be preferably used.

The compounds of Formula II-CC of Scheme 2 may be prepared as shown below in Scheme 3.

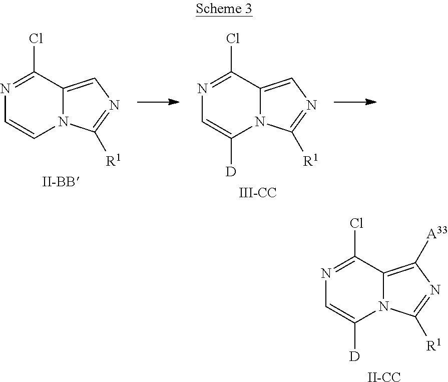

R¹ is as defined previously for compound of Formula I and A³³=halogen such as Cl, Br, or I.

In a typical preparation of a compound of Formula II-CC, intermediate II-BB′ may be converted to compound of Formula III-CC first. Intermediate of Formula II-BB′ may be treated with a strong base in a suitable solvent at a suitable reaction temperature, then the reaction may be quenched with a suitable deuterium source. Suitable bases for use in the above process may include, but are not limited to, n-BuLi, s-BuLi, t-BuLi, LDA, LiTMP and the like. Suitable solvents for use in the above process may include, but are not limited to, ethers such as THF, glyme, and the like. If desired, mixtures of these solvents may be used. The preferred solvents may be THF. Additives such as, but not limited to, HMPA (hexamethylphosphoramide) or TMEDA. (tetramethylethylenediamine) and the like may be added if necessary. The above process may be carried out at temperatures between about −100° C. and about 120° C. Preferably, the reaction may be carried out between −90° C. and about 0° C. Suitable deuterium sources for use in the above process may include, but are not limited to, D₂O, CD₃OD and the like. The preferred deuterium source may be CD₃OD. The above process to produce compounds of the present invention may be preferably carried out at about atmospheric pressure although higher or lower pressures may be used if desired. Substantially, equimolar amounts of reactants may be preferably used although higher or lower amounts may be used if desired.

In the conversion of compound of Formula III-CC to II-CC, suitable halogenating agent may be used, but are not limited to, Br₂, I₂, Cl₂, N-chlorosuccinimide, N-bromosuccinimide, or N-iodosuccinimide. The preferred halogenating agent may be N-iodosuccinimide. Suitable solvents for use in the above process may include, but are not limited to, ethers such as THF, glyme, and the like; DMF; DMSO; MeCN; alcohols such as MeOH, EtOH, isopropanol, trifluoroethanol, and the like; and chlorinated solvents such as DCM or chloroform (CHCl₃). If desired, mixtures of these solvents may be used, however, the preferred solvent may be DMF. The above process may be carried out at temperatures between about −78° C. and about 120° C. Preferably, the reaction may be carried out between 0° C. and about 75° C. The above process to produce compounds of the present invention may be preferably carried out at about atmospheric pressure although higher or lower pressures may be used if desired. Substantially, equimolar amounts of reactants may be preferably used although higher or lower amounts may be used if desired.

Scheme 4-6 generally describes incorporation of deuterium in an imidazo[5,1-f][1,2,4]triazine core followed by halogenation, ammonolysis and installation of Q¹ group.

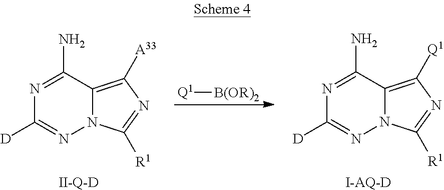

Q¹ and R¹ are as defined previously for compound of Formula I, A³³=halogen such as Cl, Br, or I; B(OR)₂=suitable boronic acid/esters. In a typical preparation of compounds of Formula I-AQ-D, compound of Formula II-Q-D maybe reacted with a suitable boronic acid/ester (Q¹-B(OR)₂) in a suitable solvent via typical Suzuki coupling procedures. Suitable solvents for use in the above process may include, but are not limited to, water, ethers such as THF, glyme, and the like; DMF; DMSO; MeCN; alcohols such as MeOH, EtOH, isopropanol, trifluoroethanol, and the like; and chlorinated solvents such as DCM or chloroform (CHCl₃). If desired, mixtures of these solvents may be used, however, the preferred solvent is glyme/water. The above process may be carried out at temperatures between about −78° C. and about 120° C. Preferably, the reaction may be carried out between 80° C. and about 100° C. The above process to produce compounds of the present invention is preferably carried out at about atmospheric pressure although higher or lower pressures may be used if desired. Substantially equimolar amounts of reactants may be used although higher or lower amounts may be used if desired.

One skilled in the art will appreciate that alternative methods may be applicable for preparing compounds of Formula I-AQ-D from II-Q-D. For example, compound of Formula II-Q-D could be reacted with a suitable organotin reagent Q¹-SnBu₃ or the like in a suitable solvent via typical Stille coupling procedures.

The compounds of Formula II-Q-D of Scheme 4 may be prepared as shown below in Scheme 5.

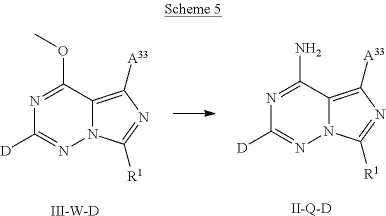

Q¹ and R¹ are as defined previously for compound of Formula I, and A³³=halogen such as Cl, Br, or I.

In a typical preparation of compounds of Formula II-Q-D, compound of Formula III-W-D is reacted with ammonia in a suitable solvent. Suitable solvents for use in the above process may include, but are not limited to, ethers such as THF, glyme, and the like; alcohols such as MeOH, EtOH, isopropanol, trifluoroethanol, and the like; and chlorinated solvents such as DCM or chloroform (CHCl₃). If desired, mixtures of these solvents may be used. The above process may be carried out at temperatures between about 0° C. and about 50° C. Preferably, the reaction may be carried out at between 0° C. and about 22° C. The above process to produce compounds of the present invention may be preferably carried out at about atmospheric pressure although higher or lower pressures may be used if desired. Substantially equimolar amounts of reactants may be preferably used although higher or lower amounts may be used if desired.

The compounds of Formula III-W-D of Scheme 5 may be prepared as shown below in Scheme 6.

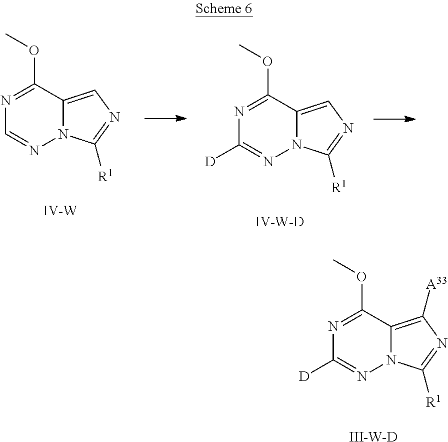

where R¹ is as defined previously for compound of Formula I and A³³=halogen such as Cl, Br, or I.

In a typical preparation of a compound of Formula III-W-D, compound IV-W may be converted to compound of Formula IV-W-D first. Compound IV-W may be treated with a strong base in a suitable solvent at a suitable reaction temperature, then the reaction may be quenched with a suitable deuterium source. Suitable bases for use in the above process may include, but are not limited to, n-BuLi, s-BuLi, t-BuLi, LDA, LiTMP and the like. Suitable solvents for use in the above process may include, but are not limited to, ethers such as THF, glyme, and the like, if desired, mixtures of these solvents may be used. The preferred solvents may be THF. Additives such as, but not limited to, HMPA (hexamethylphosphoramide) or TMEDA. (tetramethylethylenediamine) and the like may be added if necessary. The above process may be carried out at temperatures between about −100° C. and about 120° C. Preferably, the reaction may be carried out between −90° C. and about 0° C. Suitable deuterium sources for use in the above process may include, but are not limited to, D₂O, CD₃OD and the like. The preferred deuterium source may be CD₃OD. The above process to produce compounds of the present invention may be preferably carried out at about atmospheric pressure although higher or lower pressures may be used if desired. Substantially, equimolar amounts of reactants may be preferably used although higher or lower amounts may be used if desired.

Compounds of Formula III-W-D may be prepared by reacting compound of Formula IV-W-D with a suitable halogenating agent. Suitable halogenating agents include, but are not limited to, Br₂, I₂, Cl₂, N-chlorosuccinimide, N-bromosuccinimide, or N-iodosuccinimide. The preferred halogenating agent may be N-iodosuccinimide. Suitable solvents for use in the above process may include, but are not limited to, ethers such as THF, glyme, and the like; DMF; DMSO; MeCN; alcohols such as MeOH, EtOH, isopropanol, trifluoroethanol, and the like; and chlorinated solvents such as DCM or chloroform (CHCl₃). If desired, mixtures of these solvents may be used, however, the preferred solvent may be DMF. The above process may be carried out at temperatures between about −78° C. and about 120° C. Preferably, the reaction may be carried out between 40° C. and about 75° C. The above process to produce compounds of the present invention may be preferably carried out at about atmospheric pressure although higher or lower pressures may be used if desired. Substantially, equimolar amounts of reactants may be preferably used although higher or lower amounts may be used if desired.

The group Q¹ of Formula I may be prepared with various deuterium incorporations as desired.

Scheme 7-11 describes the general preparation of deuterated heteroaromatic intermediates for incorporation as Q¹.

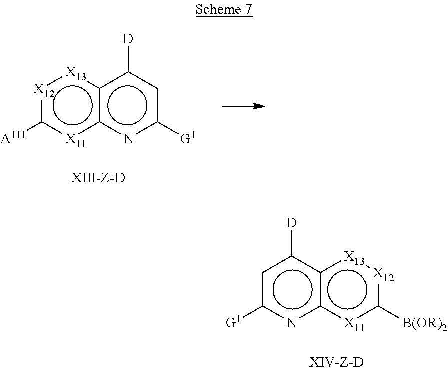

G¹, X₁₁, X₁₂, X₁₃ are as defined previously for compound of Formula I, A¹¹¹=OTf or halogen such as Cl and B(OR)₂=suitable boronic acid/esters.

In a typical preparation of a compound of Formula XIV-Z-D, a compound of Formula XIII-Z-D may be reacted with a suitable metal catalyst and a suitable boronating agent under suitable reaction conditions. Suitable metal catalyst agents may include, but are not limited to, Pd(OAc)₂ in the presence of 1,3-bis(2,6-diisopropylphenyl)imidazolium chloride. Suitable boronating agents may include, but are not limited to, bis(pinacolato)diboron. Suitable reaction conditions for use in the above process may include, but are not limited to, heating a mixture of Pd(OAc)₂, 1,3-bis(2,6-diisopropylphenyl)imidazolium chloride, KOAc, and bis(pinacol)borane in a suitable solvent such as, but not limited to, THF. The above process may be carried out at temperatures between about 20° C. and about 100° C. Preferably, the reaction may be carried out at 60° C. to 80° C. The above process to produce compounds of the present invention may be preferably carried out at about atmospheric pressure although higher or lower pressures may be used if desired. Higher or lower equivalents of reagents may be used if desired. Additionally, other suitable reaction conditions for the conversion of XIII-Z-D to XIV-Z-D can be found in the literature which involve a variety of aryl/heteroarylhalides and a variety of conditions (Biooganic & Medicinal Chemistry Letters, 2003, 12(22), 4001; Biooganic & Medicinal Chemistry Letters, 2003, 13(18), 3059; Chemical Communications (Cambridge, UK), 2003, 23, 2924; Synthesis, 2002, 17, 2503; Angewandte Chemie, International Ed., 2002, 41(16), 3056; Journal of the American Chemical Society, 2002, 124(3), 390; Organic Letters, 2002, 4(4), 541; Tetrahedron, 2001, 57(49), 9813; Journal of Organic Chemistry, 2000, 65(1), 164; Journal of Organic Chemistry, 1997, 62(19), 6458; Journal of Organometallic Chemistry, 1983, 259(3), 269).

The compounds of Formula XIII-Z-D of Scheme 7 may be prepared as shown below in Scheme 8.

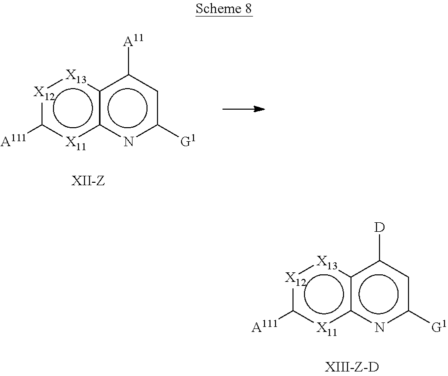

G¹, X₁₁, X₁₂, X₁₃ are as defined previously for compound of Formula I, A¹¹¹=OTf or halogen such as Cl. A¹¹=Br or I.

In a typical preparation of a compound of Formula XIII-Z-D, compound XII-Z may be subjected to metal-Halogen exchange in a suitable solvent at a suitable reaction temperature, followed by quenching with a suitable deuterium source. Suitable metal reagents for use in the above process may include, but are not limited to, n-BuLi, s-BuLi, t-BuLi, iPrMgCl and the like. Suitable solvents for use in the above process may include, but are not limited to, ethers such as THF, glyme, and the like, if desired, mixtures of these solvents may be used. The preferred solvents may be THF. The above process may be carried out at temperatures between about −100° C. and about 120° C. Preferably, the reaction may be carried out between −90° C. and about 0° C. Suitable deuterium sources for use in the above process may include, but are not limited to, D₂O, CD₃OD and the like. The preferred deuterium source may be CD₃OD. The above process to produce compounds of the present invention may be preferably carried out at about atmospheric pressure although higher or lower pressures may be used if desired. Substantially, equimolar amounts of reactants may be preferably used although higher or lower amounts may be used if desired.

The compounds of Formula XII-Z of Scheme 8 may be prepared as shown below in Scheme 9.

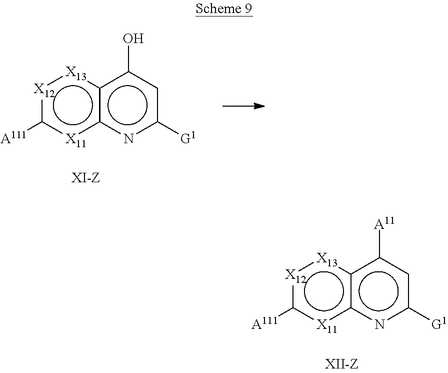

G¹, X₁₁, X₁₂, X₁₃ are as defined previously for compound of Formula I, A¹¹¹=OTf or halogen such as Cl, A¹¹=Br or I.

In a typical preparation of a compound of Formula XII-Z, where A¹¹=Br, compound XI-Z may be treated with POBr₃ in a suitable solvent at a suitable reaction temperature. Suitable solvents for use in the above process may include, but are not limited to, ethers such as THF, glyme, and the like; MeCN; and chlorinated solvents such as DCM or chloroform (CHCl₃). The preferred solvents may include DCM and MeCN. If desired, mixtures of these solvents may be used. The above process may be carried out at temperatures between about −10° C. and about 120° C. Preferably, the reaction may be carried out between 25° C. and about 100° C. The above process to produce compounds of the present invention may be preferably carried out at about atmospheric pressure although higher or lower pressures may be used if desired. Substantially, equimolar amounts of reactants may be preferably used although higher or lower amounts may be used if desired.

One skilled in the art will appreciate the methods described in literature may be applicable for preparing compounds of Formula XII-Z (where A¹¹=I) from compound XI-Z. Such methods could be found in Monatshefte für Chemie 2008,139(2), 179-181; Chemical Communications 2006, (45), 4744-4746; Tetrahedron 2005, 61(7), 1755-1763; Journal of Heterocyclic Chemistry 1977,14(3), 435-438.

The compounds of Formula XI-Z of Scheme 9 may be prepared as shown below in Scheme 10.

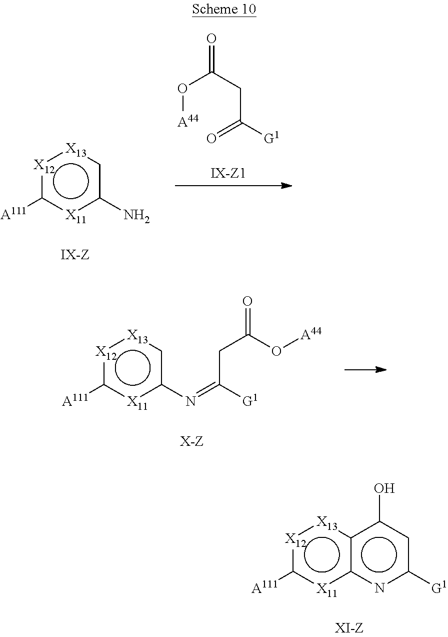

G¹, X₁₁, X₁₂, X₁₃ are as defined previously for compound of Formula I, A¹¹¹=OTf or halogen such as Cl, A⁴⁴ is alkyl groups such as methyl or ethyl.

In a typical preparation of a compound of Formula XI-Z, compound IX-Z may be reacted with compound IX-Z1 in a suitable solvent at a suitable reaction temperature using a suitable acid as catalyst to produce compound X-Z first. Suitable solvents for use in the above process may include, but are not limited to, benzene and toluene. If desired, mixtures of these solvents may be used. A Dean-Stark apparatus may be used for this reaction to remove water from reaction mixture. Suitable acid catalysts may include, but are not limited to, p-toluenesulfonic acid, HCl, H₂SO₄ and the like or pyridinium p-toluenesulfonate. The preferred acid catalyst may be p-toluenesulfonic acid. The above process may be carried out at temperatures between about 0° C. and about 150° C. Preferably, the reaction may be carried out between 60° C. and about 120° C. The above process to produce compounds of the present invention may be preferably carried out at about atmospheric pressure although higher or lower pressures may be used if desired. Substantially, equimolar amounts of reactants may be preferably used although higher or lower amounts may be used if desired.

Compound X-Z may be then treated with a suitable acid at a suitable reaction temperature to produce compound XI-Z. Reaction may be carried out using minimum amount of solvent or at neat condition. Suitable acids may include, but are limited to, polyphosphoric acid or H₂SO₄ and the like. Suitable solvents for use in the above process may include, but are not limited to, ethers such as THF, glyme, and the like; MeCN; DMF; toluene and the like; and chlorinated solvents such as DCM or chloroform (CHCl₃). If desired, mixtures of these solvents may be used. The above process may be carried out at temperatures between about −0° C. and about 200° C. Preferably, the reaction may be carried out between 25° C. and about 180° C. The above process to produce compounds of the present invention may be preferably carried out at about atmospheric pressure although higher or lower pressures may be used if desired. Substantially, equimolar amounts of reactants may be preferably used although higher or lower amounts may be used if desired.

The compounds of Formula XI-Z of Scheme 9 may also be prepared as shown below in Scheme 11.

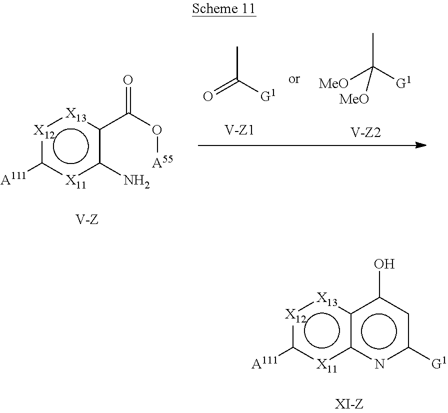

G¹, X₁₁, X₁₂, X₁₃ are as defined previously for compound of Formula I, A¹¹¹=OTf or halogen such as Cl, A⁵⁵ is a proton or alkyl group such as methyl or ethyl.

In a typical preparation of a compound of Formula XI-Z, compound V-Z may be reacted with compound V-Z1 in a suitable solvent at a suitable reaction temperature. Suitable solvents for use in the above process may include, but are not limited to, toluene and the like; diphenyl ether. If desired, mixtures of these solvents may be used. The above process may be carried out at temperatures between about 0° C. and about 250° C. Preferably, the reaction may be carried out between 100° C. and about 250° C. The above process to produce compounds of the present invention may be preferably carried out at about atmospheric pressure although higher or lower pressures may be used if desired. Substantially, equimolar amounts of reactants may be preferably used although higher or lower amounts may be used if desired.

One skilled in the art will appreciate that compounds of Formula XI-Z may also be prepared by treating compounds of Formula V-Z with compounds of Formula V-Z2 under similar reaction condition described above.

Both R¹ and Q¹ in the compounds described herein in some instances contain functional groups which can be further manipulated. It would be appreciated by those skilled in the art that such manipulation of functional groups can be accomplished with key intermediates or with late stage compounds.

The Group G¹ (of Q¹) can itself have deuterium incorporation. As described in Scheme 15 of US 2006/0235031, G¹ can then be incorporated in the larger Q¹ by organolithium or Grignard followed by oxidation. Preparations 1-4 below, illustrate incorporation of deuterium in a Q¹ moiety.

The incorporation of R¹ and other upstream transformations are known as described, e.g., in US2006/0235031. R¹ can be prepared and/or deuterated prior to incorporation in the core or in a subsequent reaction. Preparations 7-8, below, illustrate Grignard incorporation of deuterium in R¹.

The following Preparations describe intermediate preparations and chemistries that may be more generally applicable to the preparation of the invention compounds.

### Preparation 1

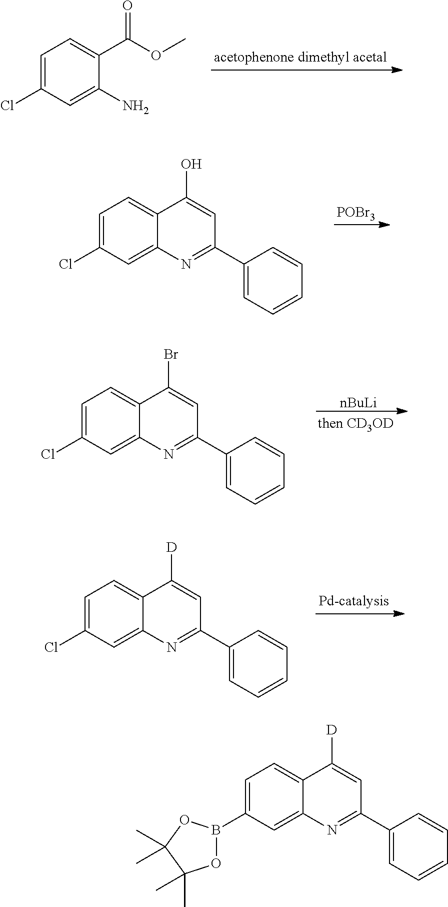

### 7-Chloro-4-hydroxy-2-phenylquinoline

A mixture of methyl 4-chloroanthranilate (27.5 g, 150 mmole), acetophenone dimethyl acetal (25 g, 150 mmole) and diphenyl ether (200 mL) was placed in a three necked 1 L round bottomed flask and through it was bubbled a stream of nitrogen gas. The temperature of the reaction mixture was maintained at 120° C. for 30 minutes, at 200° C. for 30 minutes and finally at the boiling point (250° C.) for 10 h. The flow of the nitrogen was discontinued when the reflux temperature was reached. The reaction mixture was cooled to room temperature, added hexane (100 mL) and stirred at room temperature for 30 minutes. The separated solid was filtered, washed with hexane and dried in vacuum oven at 50-55° C. Yield 26.4 g (70%). ¹H NMR (DMSO-d₆, 400 MHz) 11.72 (br s, 1H), 8.12 (d, 1H), 7.80 (m, 3H), 7.59 (m, 3H), 7.38 (d, 1H), 6.38 (s, 1H).

### 4-Bromo-7-chloro-2-phenylquinoline

To a mixture of POBr₃ (56.3 g, 196.5 mmole) in dichloroethane (250 mL) was added 7-chloro-4-hydroxy-2-phenylquinoline (21.83 g, 85.4 mmole) followed by DMF (7.02 mL, 90.7 mmole). The mixture was heated to reflux overnight. Thick solid separated out from the reaction mixture. It was cooled to room temperature and poured into ice cold ammonium hydroxide (110 mL+500 g ice) followed by methylene chloride (300 mL). The reaction mixture was stirred for 1 h, Added more methylene chloride (100 mL) to dissolve the insoluble solid. The organic layer was separated, washed with brine, dried over Na₂SO₄, filtered and concentrated. The crude solid crystallized from ethyl acetate to give 1^{st} crop 18 g. Mother liquors evaporated and crystallized from 25% ethyl acetate in hexane to give a second crop of 4 g. Again mother liquors treated with charcoal, crystallized from 25% ethyl acetate and hexane to give a 3^{rd} crop of 1 g. Total yield 23 g (74%) ¹H NMR (CDCl₃, 400 MHz) 8.13 (m, 5H), 7.52 (m, 4H).

### 7-chloro-2-phenyl-(quinoline-4-d)

A stirred solution of 4-bromo-7-chloro-2-phenylquinoline (318 mg, 1.0 mmole) in anhydrous THF (10 mL) may be treated with n-BuLi (0.44 mL, 2.5 M in hexane, Aldrich) at −100° C. for 20 min. Then the reaction may be quenched with CD₃OD (10.00 eq., 99.96 atom % D, Aldrich) at −100° C. and stirred at same temperature for 20 min before warm up to at −20° C. in 1-2 h. Saturated aq. NH₄Cl solution (5 mL) may be added to the mixture, the bulk of solvent may be removed under reduced pressure to give a residue, which may be further purified by silica gel flash chromatography (1-10% ethyl acetate in hexane).

### 2-Phenyl-7-(4,4,5,5-tetramethyl-[1,3,2]dioxaborolan-2-yl)-(quinoline-4-d)

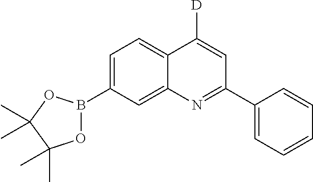

This compound may be prepared from 7-chloro-2-phenyl-(quinoline-4-d) according to similar procedure described for synthesis of 2-phenyl-7-(4,4,5,5-tetramethyl-[1,3,2]dioxaborolan-2-yl)-quinoline from 7-chloro-2-phenyl-quinoline under palladium catalysis.

### Preparation 2

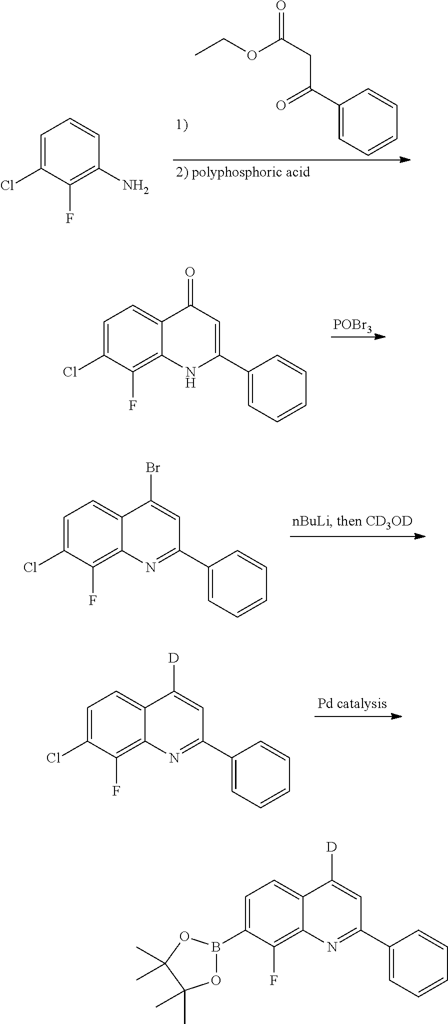

### 8-Fluoro-2-phenyl-7-(4,4,5,5-tetramethyl-[1,3,2]dioxaborolan-2-yl)-(quinoline-4-d)

This compound may be prepared from 7-chloro-8-fluoro-2-phenyl-(quinoline-4-d) according to similar procedure described for synthesis of 2-phenyl-7-(4,4,5,5-tetramethyl-[1,3,2]dioxaborolan-2-yl)-quinoline from 7-chloro-2-phenyl-quinoline under palladium catalysis.

### 7-Chloro-8-fluoro-2-phenyl-(quinoline-4-d)

A stirred solution of 4-bromo-7-chloro-8-fluoro-2-phenyl-quinoline (336 mg, 1.0 mmole) in anhydrous THF (10 mL) may be treated with n-BuLi (0.44 mL, 2.5 M in hexane, Aldrich) at −100° C. for 20 min. Then the reaction may be quenched with CD₃OD (10.00 eq., 99.96 atom % D, Aldrich) at −100° C. and stirred at same temperature for 20 min before warm up to at −20° C. in 1-2 h. Saturated aq. NH₄Cl solution (5 mL) may be added to the mixture, the bulk of solvent may be removed under reduced pressure to give a residue, which may be further purified by silica gel flash chromatography (1-10% ethyl acetate in hexane).

### 4-Bromo-7-chloro-8-fluoro-2-phenyl-quinoline

Phosphorus oxybromide (19 g, 0.066 mole), 7-chloro-8-fluoro-2-phenyl-1H-quinolin-4-one (6.2 g, 0.022 mole) and acetonitrile (40 mL) were combined in a 150 mL pressure bottle with a magnetic stir bar. The flask was heated at 100° C. and stirred overnight. Heat was removed, and an ice-water bath was installed. After 10 minutes, the bottle was opened, and water (60 mL) was added to the cooled stirring reaction. The quenching was exothermic to ca. 50° C. After stirring 10 minutes, a nice, filterable solid had formed, however, the reaction was extracted with 100 mL methylene chloride. The extracts were combined, washed with saturated sodium bicarbonate solution (100 mL), and suction filtered through a small pad of silica gel, rinsing with methylene chloride. The filtrate was concentrated in vacuo, and put under high vacuum at 45° C. for 1 h to afford the crude product, which was recrystallized from 100 mL of ethanol, suction filtered to collect, and washed with ethanol. The purified product was vacuum oven dried for 1 h at 45° C. to afford the title compound as a white solid. A second crop was taken. The mother liquor was concentrated then chromatographed on silica gel with hexanes/methylene chloride 1:1 and combined with the second crop to afford additional material; ¹H NMR (CDCl₃, 400 MHz) δ 7.49-7.55 (m, 4H), 7.87-7.90 (dd, 1H, J=1.7 Hz & J=8.9 Hz), 8.15-8.18 (dd, 2H, J=1.5 Hz & J=7.9 Hz), 8.21 (s, 1H); HPLC t_{R}=4.15 min (OpenLynx, nonpolar_{—}5 min).

### 7-Chloro-8-fluoro-2-phenyl-1H-quinolin-4-one

3-(3-Chloro-2-fluoro-phenylamino)-3-phenyl-acrylic acid ethyl ester (9.2 g, 0.029 mole) and polyphosphoric acid (160 mL, 3.0 mole) were combined and mechanically stirred under nitrogen at 175° C. external temperature for 40 minutes. While still hot, the reaction was poured over 800 mL of stirring ice-water rinsing with water. The mixture was a fine suspension, and was allowed to stir overnight. After stirring overnight, the mixture was filtered to collect the solid. The solid was washed with 4×150 mL water, and then with 4×150 mL of 4:1 ether/methanol. The solid was placed in the vacuum oven at 45° C. for 4 h and afforded the title compound as an off white product; ¹H NMR (DMSO-d₆, 400 MHz) δ 6.58 (bs, 1H), 7.50-7.54 (d of d, 1H, J=6.6 Hz & J=8.9 Hz), 7.60-7.64 (m, 3H), 7.82-7.84 (m, 2H), 8.07-8.09 (d of d, 1H, J=1.5 Hz & J=8.7 Hz); MS (ES+): 274.03 (100) [MH+], 275.99 (30) [(M+2)H+]. LCMS t_{R}=2.97 min (OpenLynx, polar_{—}5 min).

### 3-(3-Chloro-2-fluoro-phenylamino)-3-phenyl-acrylic acid ethyl ester

2-Fluoro-3-chloroaniline (7.55 mL, 0.0687 mole), ethyl benzoylacetate (13.2 g, 0.0687 mole) and p-toluenesulfonic acid (1.18 g, 0.007 mole) were combined in a 250 mL round bottom flask with toluene (60 mL) and a magnetic stir-bar. The reaction was stirred at reflux with a Dean-Stark water trap. Reflux was stopped after 3 h. The product mixture was allowed to cool, and was then passed through a short pad of silica gel with methylene chloride, and concentrated in vacuo. Standing under high vacuum for 1 h afforded an oil. The oil was stirred in 100 mL of hexanes overnight, then suction filtered to remove a solid impurity. The filtrate was concentrated, and put on high vacuum to afford an oil. The oil was chromatographed with hexanes, ethyl acetate (8:1), and put under high vacuum for 1 h to afford the title compound as a yellow oil; ¹H NMR (CDCl₃, 400 MHz) δ 1.30-1.34 (t, 3H, J=7.1 Hz), 4.20-4.25 (Q, 2H, J=7.1 Hz), 5.13 (s, 1H), 6.19-6.23 (t, 1H), 6.60-6.65 (t of d, 1H, J=1.7 & J=8.2), 6.88-6.92 (t of d, 1H, J=1.5 & J=6.6), 7.29-7.37 (m, 5H), 10.21 (bs, 1H); MS (ES⁺): 319.99, 322.02.

### Preparation 3

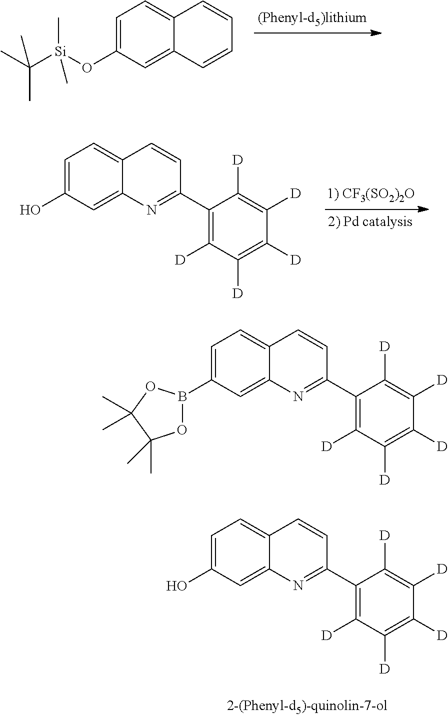

This compound may be synthesized by treating 7-(tert-butyldimethylsilyloxy)quinoline (1.0 eq.) with (phenyl-d₅) lithium (1.0-2.0 eq.) according to method described for synthesis of 2-phenylquinolin-7-ol from 7-(tert-butyldimethylsilyloxy)quinoline.

It would be appreciated by those skilled in the art that (Phenyl-d₅) lithium may be prepared in situ by adding n-BuLi (1.0 eq, 2.5 M in hexane, Aldrich) to a stirred solution of bromobenzene-d5 (1.05 eq, 0.20 M) in anhydrous THF at −100° C. and stirred for 10 min at same temperature before being used.

### 2-(Phenyl-d₅)-7-(4,4,5,5-tetramethyl-[1,3,2]dioxaborolan-2-yl)-quinoline

This compound may be synthesized from 2-(Phenyl-d₅)-quinolin-7-ol according to method described for synthesis of 2-Phenyl-7-(4,4,5,5-tetramethyl-[1,3,2]dioxaborolan-2-yl)-quinoline from 2-Phenyl-quinolin-7-ol.

### Preparation 4

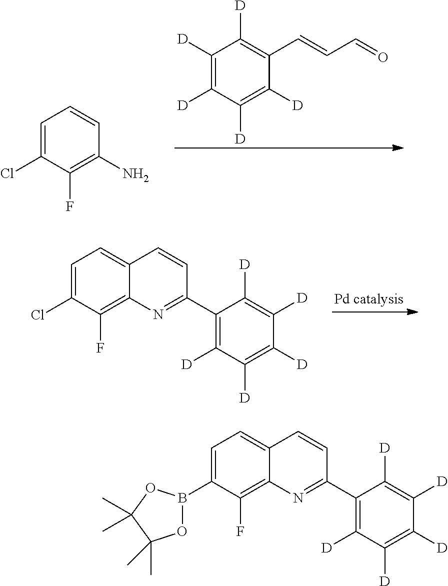

### 8-Fluoro-2-(phenyl-d₅)-7-(4,4,5,5-tetramethyl-[1,3,2]dioxaborolan-2-yl)-quinoline

This compound may be prepared from 7-chloro-8-fluoro-2-(phenyl-d₅)-quinoline according to similar procedure described for synthesis of 2-phenyl-7-(4,4,5,5-tetramethyl-[1,3,2]dioxaborolan-2-yl)quinoline from 7-chloro-2-phenyl-quinoline under palladium catalysis.

### 7-chloro-8-fluoro-2-(phenyl-d₅)-quinoline

This compound may be prepared from 3-chloro-2-fluoroaniline and (E)-3-(phenyl-d₅)-propenal according to similar procedure described for synthesis of 7-chloro-8-fluoro-2-phenyl-quinoline from 3-chloro-2-fluoroaniline and trans-cinnamaldehyde.

### (E)-3-(Phenyl-d₅)-propenal

This compound may be synthesized from benzaldehyde-2,3,4,5,6-d₅ (CAS#: 14132-51-5) according to methods described in literature for synthesis of trans-cinnamaldehyde from benzaldehyde. Some representative methods are described in Synthetic Communications, 37(21), 2007, 3841-3854; Journal of Organic Chemistry, 72(21), 2007, 7974-7979; Organic & Biomolecular Chemistry, 4(15), 2006, 2912-2927; Synlett, (9), 2000, 1345-1347.

### Preparation 5

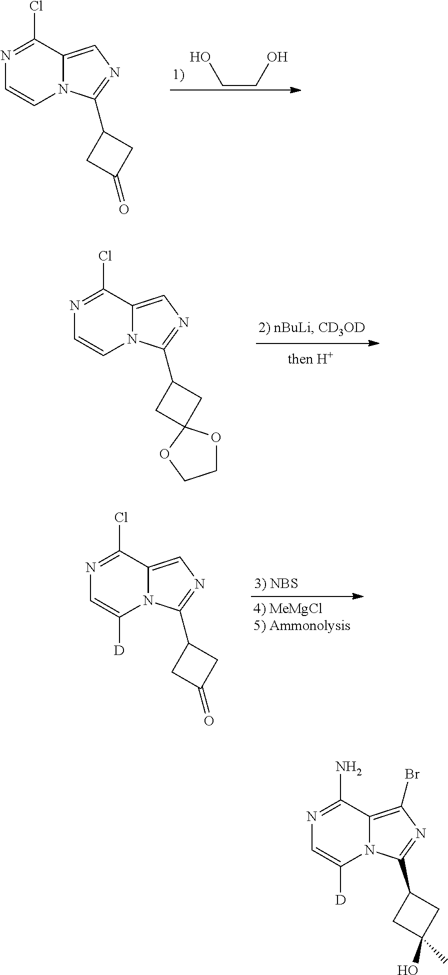

### 8-Chloro-3-(5,8-dioxa-spiro[3.4]oct-2-yl)-imidazo[1,5-a]pyrazine

A solution of 3-(8-chloro-imidazo[1,5-a]pyrazin-3-yl)cyclobutanone (0.55 g, 2.5 mmol), p-TsOH.H₂O (24.7 mg, 0.13 mmol), ethylene glycol (1.0 mL) in benzene (15 mL) may be refluxed under a Dean-Stark trap for about 3 h. The mixture may be then cooled to room temperature, diluted with ether, and wash with saturated NaHCO₃ aqueous solution and brine. The organic phase may be dried over MgSO₄ and concentrated. The residue may be purified through a short column of silica gel (1-10% AcOEt in hexane) to afford desired product.

### 3-(8-Chloro-imidazo[1,5-a]pyrazin-5-d-3-yl)-cyclobutanone

A stirred solution of 8-chloro-3-(5,8-dioxa-spiro[3.4]oct-2-yl)-imidazo[1,5-a]pyrazine (265 mg, 1.0 mmole) in anhydrous THF (10 mL) may be treated with n-BuLi (0.44 mL, 2.5 M in hexane, Aldrich) at −78° C. for 20 min. Then the reaction may be quenched with CD₃OD (10.00 eq., 99.96 atom % D, Aldrich) at −78° C. and stirred at same temperature for 20 min before warm up to at −20° C. in 1-2 h. Saturated aq. NH₄Cl solution (5 mL) may be added to the mixture, The bulk of solvent may be removed under reduced pressure to give a residue, which may be diluted with acetonitrile (3 mL) and 2N sulfuric acid (1.5 mL). The mixture may be stirred at room temperature for 16-24 h. The reaction may be then quenched by neutralization with aqueous NaHCO₃, and the mixture may be extracted with DCM and washed with brine. The extract may be dried over Na₂SO₄ and evaporated to afford a residue which may be further purified by silica gel flash chromatography (1-10% ethyl acetate in hexane) to give the titled compound.

### cis-3-(8-Amino-1-bromo-(imidazo[1,5-a]pyrazin-5-d)-3-yl)-1-methyl-cyclobutanol

This compound may be synthesized from 3-(8-chloro-(imidazo[1,5-a]pyrazin-5-d)-3-yl)-cyclobutanone according to the procedure described for synthesis of cis-3-(8-amino-1-bromo-imidazo[1,5-a]pyrazin-3-yl)-1-methyl-cyclobutanol from 3-(8-chloro-imidazo[1,5-a]pyrazin-3-yl)-cyclobutanone.

### Preparation 6

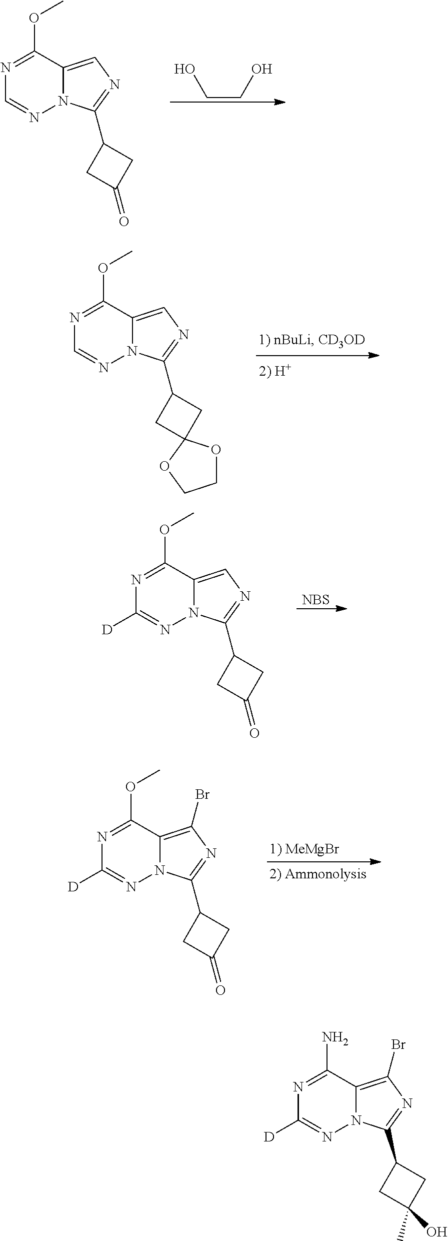

### cis-3-(4-Amino-5-bromo-(imidazo[5,1-f][1,2,4]triazin-2-d)-7-yl)-1-methyl-cyclobutanol

This compound may be synthesized from 3-(4-methoxy-(imidazo[5,1-f][1,2,4]triazin-2-d)-7-yl)cyclobutanone according to the procedures described for synthesis of cis-3-(4-amino-5-bromo-imidazo[5,1f][1,2,4]triazin-7-yl)-1-methyl-cyclobutanol from 3-(4-methoxy-imidazo[5,1f][1,2,4]triazin-7-yl)cyclobutanone.

### 3-(4-Methoxy-(imidazo[5,1f][1,2,4]triazin-2-d)-7-yl)cyclobutanone

A stirred solution of 7-(5,8-dioxa-spiro[3.4]oct-2-yl)-4-methoxy-imidazo[5,1-f][1,2,4]triazine (262 mg, 1.0 mmole) in anhydrous THF (10 mL) may be treated with n-BuLi (0.44 mL, 2.5 M in hexane, Aldrich) at −78° C. for 20 min. Then the reaction may be quenched with CD₃OD (10.00 eq., 99.96 atom % D, Aldrich) at −78° C. and stirred at same temperature for 20 min before warm up to at −20° C. in 1-2 h. Saturated aq. NH₄Cl solution (5 mL) may be added to the mixture, The bulk of solvent may be removed under reduced pressure to give a residue, which may be diluted with acetonitrile (3 mL) and 2N sulfuric acid (1.5 mL). The mixture may be stirred at room temperature for 16-24 h. The reaction may be then quenched by neutralization with aqueous NaHCO₃, and the mixture may be extracted with DCM and washed with brine. The extract may be dried over Na₂SO₄ and evaporated to afford a residue which may be further purified by silica gel flash chromatography (1-10% ethyl acetate in hexane) to give the titled compound.

### 7-(5,8-Dioxa-spiro[3.4]oct-2-yl)-4-methoxy-imidazo[5,1f][1,2,4]triazine

A solution of 3-(4-methoxy-imidazo[5,1f][1,2,4]triazin-7-yl)cyclobutanone (0.55 g, 2.5 mmol), p-TsOH.H₂O (24.7 mg, 0.13 mmol), ethylene glycol (1.0 mL) in benzene (15 mL) may be refluxed under a Dean-Stark trap for about 3 h. The mixture may be then cooled to room temperature, diluted with ether, and wash with saturated NaHCO₃ aqueous solution and brine. The organic phase may be dried over MgSO₄ and concentrated. The residue may be purified through a short column of silica gel (1-10% ethyl acetate in hexane) to afford desired product.

### Preparation 7

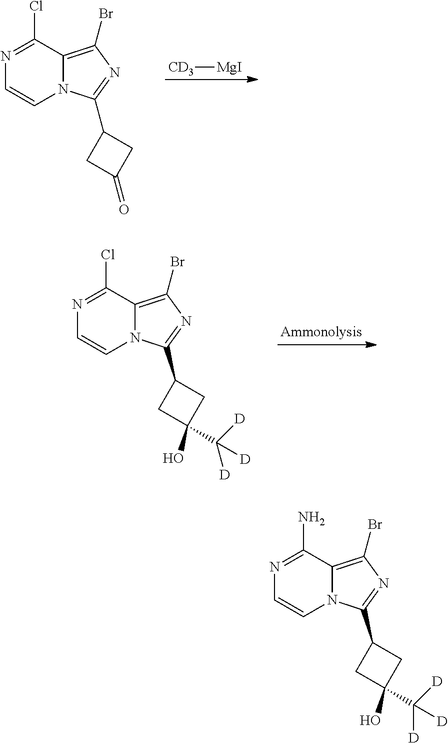

### cis-3-(1-Bromo-8-chloroimidazo[1,5-a]pyrazin-3-yl)-1-(methyl-d₃)-cyclobutanol

3-(1-Bromo-8-chloroimidazo[1,5-a]pyrazin-3-yl)cyclobutanone (5.2 g, 0.017 mol) in anhydrous THF (60 mL) under nitrogen at −78° C. may be treated with a 1.0 M solution of methyl-d₃ magnesium Iodide in diethyl ether (99 atom % D, 38 mL, 0.038 mol) over 30 min. The mixture may be stirred at −78° C. for 30 min and then the cooling bath may be removed and the mixture may be quenched with saturated aq. NH₄Cl. EtOAc may be added to the aqueous phase. The combined organic phases may be concentrated in vacuo to give a crude residue which may be purified by flash chromatography (1-20% ethyl acetate in hexane) to give the titled compound.

### cis-3-(8-Amino-1-bromoimidazo[1,5-a]pyrazin-3-yl)-1-(methyl-d₃)-cyclobutanol

This compound may be synthesized from cis-3-(1-bromo-8-chloroimidazo[1,5-a]pyrazin-3-yl)-1-(methyl-d₃)-cyclobutanol according to the method described for synthesis of cis-3-(8-amino-1-bromoimidazo[1,5-a]pyrazin-3-yl)-1-methylcyclobutanol from cis-3-(1-bromo-8-chloroimidazo[1,5-a]pyrazin-3-yl)-1-methyl-cyclobutanol.

### Preparation 8

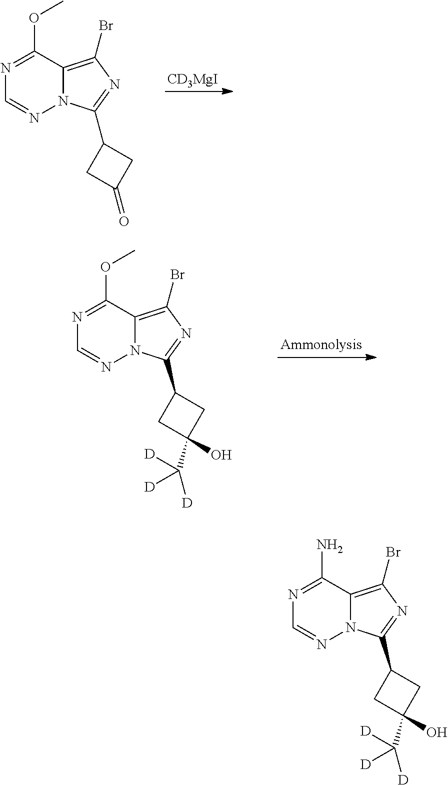

### cis-3-(4-Amino-5-bromo-imidazo[5,1-f][1,2,4]triazin-7-yl)-1-(methyl-d₃)-cyclobutanol

This compound may be synthesized from cis-3-(5-bromo-4-methoxy-imidazo[5,1-f][1,2,4]triazin-7-yl)-1-(methyl-d₃)-cyclobutanol according to the method described for synthesis of cis-3-(4-amino-5-bromo-imidazo[5,1-f][1,2,4]triazin-7-yl)-1-methyl-cyclobutanol from cis-3-(5-bromo-4-methoxy-imidazo[5,1-f][1,2,4]triazin-7-yl)-1-methyl-cyclobutanol.

### cis-3-(5-Bromo-4-methoxy-imidazo[5,1-f][1,2,4]triazin-7-yl)-1-(methyl-d₃)-cyclobutanol

This compound may be synthesized from 3-(5-bromo-4-methoxy-imidazo[5,1-f][1,2,4]triazin-7-yl)cyclobutanone using CD₃MgI according to the procedure described for synthesis of cis-3-(5-bromo-4-methoxy-imidazo[5,1-f][1,2,4]triazin-7-yl)-1-methyl-cyclobutanol from 3-(5-bromo-4-methoxy-imidazo[5,1-f][1,2,4]triazin-7-yl)cyclobutanone.

The following useful intermediates are known in the art: cis-3-(8-Amino-1-bromoimidazo[1,5-a]pyrazin-3-yl)-1-methylcyclobutanol; 2-Phenyl-7-(4,4,5,5-tetramethyl-[1,3,2]dioxaborolan-2-yl)-quinoline; cis-3-(4-Amino-5-bromo-imidazo[5,1-f][1,2,4]triazin-7-yl)-1-methyl-cyclobutanol; 8-Fluoro-2-phenyl-7-(4,4,5,5-tetramethyl-[1,3,2]dioxaborolan-2-yl)-quinoline.

### EXAMPLES

### Example 1

### cis-3-[8-Amino-1-(2-phenyl-quinolin-7-yl)-imidazo[1,5-a]pyrazin-3-yl]-1-(methyl-d₃)-cyclobutanol

This compound may be synthesized from cis-3-(8-amino-1-bromoimidazo[1,5-a]pyrazin-3-yl)-1-(methyl-d₃)-cyclobutanol and 2-phenyl-7-(4,4,5,5-tetramethyl-[1,3,2]dioxaborolan-2-yl)-quinoline by a Pd-catalyzed Suzuki coupling reaction:

A solution of cis-3-(8-amino-1-bromoimidazo[1,5-a]pyrazin-3-yl)-1-(methyl-d₃)-cyclobutanol (3.35 g, 11.15 mmol), 2-phenyl-7-(4,4,5,5-tetramethyl-[1,3,2]dioxaborolan-2-yl)-quinoline (16.7 mmol), potassium carbonate (4.62 g, 33.4 mmol) and 1,1′-bis(diphenylphosphino)ferrocenepalladium(II) dichloride, dichloromethane (0.46 g, 0.56 mmol) in previously degassed 5:1 dioxane water (166 mL) may be heated to 90° C. for 2 h. The reaction may be monitored by TLC and/or LC-MS. The reaction mixture may be charged with additional amount of 2-phenyl-7-(4,4,5,5-tetramethyl-[1,3,2]dioxaborolan-2-yl)-quinoline and 1,1′-bis(diphenylphosphino)ferrocenepalladium(II) dichloride, dichloromethane if necessary. After the completion of reaction, the reaction mixture may be partitioned between CHCl₃ and H₂O and separated. The aqueous may be re-extracted with CHCl₃ (50 mL×3) and the combined organic fractions may be dried over Na₂SO₄, filtered and concentrated in vacuo. The crude reaction mixture may be purified by a silica gel flash chromatography (1-5% MeOH in DCM).

### Example 2

### cis-3-[8-Amino-1-(2-phenyl-(quinolin-4-d)-7-yl)-imidazo[1,5-a]pyrazin-3-yl]-1-methyl-cyclobutanol

This compound may be synthesized from cis-3-(8-amino-1-bromoimidazo[1,5-a]pyrazin-3-yl)-1-methyl-cyclobutanol and 2-phenyl-7-(4,4,5,5-tetramethyl-[1,3,2]dioxaborolan-2-yl)-(quinoline-4-d) by a Pd-catalyzed Suzuki coupling reaction according to procedures described for synthesis of example 1.

### Example 3

### cis-3-[8-Amino-1-(2-phenyl-quinolin-7-yl)-(imidazo[1,5-a]pyrazin-5-d)-3-yl]-1-methyl-cyclobutanol

This compound may be synthesized from cis-3-(8-amino-1-bromo-(imidazo[1,5-a]pyrazin-5-d)-3-yl)-1-methyl-cyclobutanol and 2-phenyl-7-(4,4,5,5-tetramethyl-[1,3,2]dioxaborolan-2-yl)-quinoline by a Pd-catalyzed Suzuki coupling reaction according to procedures described for synthesis of example 1.

### Example 4

### cis-3-[8-Amino-1-(2-(phenyl-d₅)-quinolin-7-yl)-(imidazo[1,5-a]pyrazin)-3-yl]-1-methyl-cyclobutanol

This compound may be synthesized from cis-3-(8-amino-1-bromoimidazo[1,5-a]pyrazin-3-yl)-1-methyl-cyclobutanol and 2-(phenyl-d₅)-7-(4,4,5,5-tetramethyl-[1,3,2]dioxaborolan-2-yl)-quinoline) by a Pd-catalyzed Suzuki coupling reaction according to procedures described for synthesis of example 1.

### Example 5

### cis-3-[4-Amino-5-(8-fluoro-2-phenyl-quinolin-7-yl)-imidazo[5,1-f][1,2,4]triazin-7-yl]-1-(methyl-d₃)-cyclobutanol

This compound may be synthesized from cis-3-(4-Amino-5-bromo-imidazo[5,1-f][1,2,4]triazin-7-yl)-1-(methyl-d₃)-cyclobutanol and 8-fluoro-2-phenyl-7-(4,4,5,5-tetramethyl-[1,3,2]dioxaborolan-2-yl)-quinoline by a Pd-catalyzed Suzuki coupling reaction according to procedures described for synthesis of example 1.

### Example 6

### cis-3-[4-Amino-5-(8-fluoro-2-phenyl-(quinolin-4-d)-7-yl)-imidazo[5,1-f][1,2,4]triazin-7-yl]-1-methyl-cyclobutanol

This compound may be synthesized from cis-3-(4-amino-5-bromo-imidazo[5,1-f][1,2,4]triazin-7-yl)-1-methyl-cyclobutanol and 8-fluoro-2-phenyl-7-(4,4,5,5-tetramethyl-[1,3,2]dioxaborolan-2-yl)-(quinoline-4-d) by a Pd-catalyzed Suzuki coupling reaction according to procedures described for synthesis of example 1.

### Example 7

### cis-3-[4-Amino-5-(8-fluoro-2-phenyl-quinolin-7-yl)-(imidazo[5,1-f][1,2,4]triazin-2-d)-7-yl]-1-methyl-cyclobutanol

This compound may be synthesized from cis-3-(4-amino-5-bromo-(imidazo[5,1-f][1,2,4]triazin-2-d)-7-yl)-1-methyl-cyclobutanol and 8-fluoro-2-phenyl-7-(4,4,5,5-tetramethyl-[1,3,2]dioxaborolan-2-yl)-quinoline by a Pd-catalyzed Suzuki coupling reaction according to procedures described for synthesis of example 1.

### Example 8

### cis-3-[4-Amino-5-(8-fluoro-2-(phenyl-d₅)-quinolin-7-yl)-imidazo[5,1-f][1,2,4]triazin-7-yl]-1-methyl-cyclobutanol

This compound may be synthesized from cis-3-(4-amino-5-bromo-imidazo[5,1-f][1,2,4]triazin-7-yl)-1-methyl-cyclobutanol and 8-fluoro-2-(phenyl-d₅)-7-(4,4,5,5-tetramethyl-[1,3,2]dioxaborolan-2-yl)-quinoline by a Pd-catalyzed Suzuki coupling reaction according to procedures described for synthesis of example 1.

### Biological Properties

In some aspects of the invention, compounds of the invention are inhibitors of kinases, including at least one of IGF-1R or IR.

In some aspects of the invention, compounds of the invention are selective inhibitors of IGF-1R and/or IR. In some embodiments, the compound is a selective inhibitor of IGF-1R and/or IR over other kinase targets.

In some aspects of the invention, a compound of the invention has an IGF-1R inhibitory activity in an in vitro biochemical assay with an IC₅₀ value of about 200 nM or less, or about 100 nM or less. The compounds may be assayed according to the methods disclosed in US 2006/0235031.

### Compositions

The invention includes pharmaceutical compositions comprising a compound or pharmaceutically acceptable salt thereof of the invention, which is formulated for a desired mode of administration with or without one or more pharmaceutically acceptable and useful carriers. The compounds can also be included in pharmaceutical compositions in combination with one or more other therapeutically active compounds.

The pharmaceutical compositions of the present invention comprise a compound of the invention (or a pharmaceutically acceptable salt thereof) as an active ingredient, optional pharmaceutically acceptable carrier(s) and optionally other therapeutic ingredients or adjuvants. The compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions may be conveniently presented in unit dosage form and prepared by any of the methods well known in the art of pharmacy.

Compounds of the invention can be combined as the active ingredient in intimate admixture with a pharmaceutical carrier according to conventional pharmaceutical compounding techniques. The carrier may take a wide variety of forms depending on the form of preparation desired for administration, e.g., oral or parenteral (including intravenous). Thus, the pharmaceutical compositions of the present invention can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion, or as a water-in-oil liquid emulsion. In addition to the common dosage forms set out above, the compound represented by Formula I, or a pharmaceutically acceptable salt thereof, may also be administered by controlled release means and/or delivery devices. The compositions may be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

The pharmaceutical carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

A tablet containing the composition of this invention may be prepared by compression or molding, optionally with one or more accessory ingredients or adjuvants. Compressed tablets may be prepared by compressing, in a suitable machine, the active ingredient in a free-flowing form such as powder or granules, optionally mixed with a binder, lubricant, inert diluent, surface active or dispersing agent. Molded tablets may be made by molding in a suitable machine, a mixture of the powdered compound moistened with an inert liquid diluent. Each tablet preferably contains from about 0.05 mg to about 5 g of the active ingredient and each cachet or capsule preferably containing from about 0.05 mg to about 5 g of the active ingredient.

A formulation intended for the oral administration to humans may contain from about 0.5 mg to about 5 g of active agent, compounded with an appropriate and convenient amount of carrier material which may vary from about 5 to about 95 percent of the total composition. Unit dosage forms will generally contain between from about 1 mg to about 2 g of the active ingredient, typically 25 mg, 50 mg, 100 mg, 200 mg, 300 mg, 400 mg, 500 mg, 600 mg, 800 mg, or 1000 mg.

Compounds of the invention can be provided for formulation at high purity, for example at least about 90%, 95%, or 98% pure by weight.

Pharmaceutical compositions of the present invention suitable for parenteral administration may be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present invention suitable for injectable use include sterile aqueous solutions or dispersions. Furthermore, the compositions can be in the form of sterile powders for the extemporaneous preparation of such sterile injectable solutions or dispersions. In all cases, the final injectable form must be sterile and must be effectively fluid for easy syringability. The pharmaceutical compositions must be stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

Pharmaceutical compositions of the present invention can be in a form suitable for topical use such as, for example, an aerosol, cream, ointment, lotion, dusting powder, or the like. Further, the compositions can be in a form suitable for use in transdermal devices. These formulations may be prepared, utilizing a compound represented by Formula I of this invention, or a pharmaceutically acceptable salt thereof, via conventional processing methods. As an example, a cream or ointment is prepared by admixing hydrophilic material and water, together with about 5 wt % to about 10 wt % of the compound, to produce a cream or ointment having a desired consistency.

Pharmaceutical compositions of this invention can be in a form suitable for rectal administration wherein the carrier is a solid. It is preferable that the mixture forms unit dose suppositories. Suitable carriers include cocoa butter and other materials commonly used in the art. The suppositories may be conveniently formed by first admixing the composition with the softened or melted carrier(s) followed by chilling and shaping in molds.

In addition to the aforementioned carrier ingredients, the pharmaceutical formulations described above may include, as appropriate, one or more additional carrier ingredients such as diluents, buffers, flavoring agents, binders, surface-active agents, thickeners, lubricants, preservatives (including anti-oxidants) and the like. Furthermore, other adjuvants can be included to render the formulation isotonic with the blood of the intended recipient. Compositions containing a compound described by Formula I, or pharmaceutically acceptable salts thereof, may also be prepared in powder or liquid concentrate form.

### Uses

Compounds of the invention inhibit the activity of tyrosine kinase enzymes in animals, including humans, and are useful in the treatment and/or prevention of various diseases and conditions such as hyperproliferative disorders such as cancer. In particular, compounds disclosed herein are inhibitors of IGF1R and/or IR.

In some aspects, the compound is administered orally.

In some aspects, the invention includes a method of treating cancer comprising administering to a mammal in need thereof a therapeutically effective amount of a compound or salt of the invention.

In some aspects, the invention includes a method of treating a cancer, such as those above, which is mediated at least in part by IGF1R and/or IR comprising administering to a mammal in need thereof a therapeutically effective amount of a compound or salt of the invention.

The compounds of Formula I of the present invention are useful in the treatment of a variety of cancers, including, but not limited to, solid tumor, sarcoma, fibrosarcoma, osteoma, melanoma, retinoblastoma, rhabdomyosarcoma, glioblastoma, neuroblastoma, teratocarcinoma, hematopoietic malignancy, and malignant ascites. More specifically, the cancers include, but not limited to, lung cancer, bladder cancer, pancreatic cancer, kidney cancer, gastric cancer, breast cancer, colon cancer, prostate cancer (including bone metastases), hepatocellular carcinoma, ovarian cancer, esophageal squamous cell carcinoma, melanoma, an anaplastic large cell lymphoma, an inflammatory myofibroblastic tumor, and a glioblastoma.

In some aspects, the above methods are used to treat one or more of bladder, colorectal, nonsmall cell lung, breast, or pancreatic cancer. In some aspects, the above methods are used to treat one or more of ovarian, gastric, head and neck, prostate, hepatocellular, renal, glioma, glioma, or sarcoma cancer.

Generally, dosage levels on the order of from about 0.01 mg/kg to about 150 mg/kg of body weight per day are useful in the treatment of the above-indicated conditions, or alternatively about 0.5 mg to about 7 g per patient per day. For example, inflammation, cancer, psoriasis, allergy/asthma, disease and conditions of the immune system, disease and conditions of the central nervous system (CNS), may be effectively treated by the administration of from about 0.01 to 50 mg of the compound per kilogram of body weight per day, or alternatively about 0.5 mg to about 3.5 g per patient per day.

It is understood, however, that the specific dose level for any particular patient will depend upon a variety of factors including the age, body weight, general health, sex, diet, time of administration, route of administration, rate of excretion, drug combination and the severity of the particular disease undergoing therapy.

In some aspects, the invention includes a method of treating cancer comprising administering to a mammal in need thereof a therapeutically effective amount of a compound or salt of the invention, wherein at least one additional active anti-cancer agent is used as part of the method.

### GENERAL DEFINITIONS AND ABBREVIATIONS

Unless otherwise stated, the connections of compound name moieties are at the rightmost recited moiety. That is, the substituent name starts with a terminal moiety, continues with any bridging moieties, and ends with the connecting moiety. For example, hetarylthioC₁₋₄alkyl has a heteroaryl group connected through a thio sulfur to a C₁₋₄ alkyl that connects to the chemical species bearing the substituent.

In all embodiments of this invention, the term “alkyl” includes both branched and straight chain alkyl groups. Typical alkyl groups are methyl, ethyl, n-propyl, isopropyl, n-butyl, sec-butyl, isobutyl, tert-butyl, n-pentyl, isopentyl, n-hexyl, n-heptyl, isooctyl, nonyl, decyl, undecyl, dodecyl, tetradecyl, hexadecyl, octadecyl, eicosyl, and the like.

As used herein, for example, “C₀₋₁₂alkyl” is used to mean an alkyl having 0-12 carbons—that is, 0, 1, 2, 3, 4, 5, 6, 7, 8, 9, 10, 11, or 12 carbons in a straight or branched configuration. C₀alkyl means a single covalent chemical bond when it is a connecting moiety, and a hydrogen when it is a terminal moiety.

Unless otherwise specified, the term “cycloalkyl” refers to a 3-12 carbon mono-cyclic, bicyclic, or polycyclic aliphatic ring structure, optionally substituted with for example, alkyl, hydroxy, oxo, and halo, such as cyclopropyl, methylcyclopropyl, cyclobutyl, cyclopentyl, 2-hydroxycyclopentyl, cyclohexyl, 4-chlorocyclohexyl, cycloheptyl, cyclooctyl, and the like. Cycloalkyl can be bicycloalkyl, polycycloalkyl or spiroalkyl.

The term “bicycloalkyl” and “polycycloalkyl” refer to a structure consisting of two or more cycloalkyl moieties that have two or more atoms in common. If the cycloalkyl moieties have exactly two atoms in common they are said to be “fused”. Examples include, but are not limited to, bicyclo[3.1.0]hexyl, perhydronaphthyl, and the like. If the cycloalkyl moieties have more than two atoms in common they are said to be “bridged”. Examples include, but are not limited to, bicyclo[2.2.1]heptyl (“norbornyl”), bicyclo[2.2.2]octyl, and the like.

The term “spiroalkyl” refers to a structure consisting of two cycloalkyl moieties that have exactly one atom in common. Examples include, but are not limited to, spiro[4.5]decyl, spiro[2.3]hexyl, and the like.

The term “heterobicycloalkyl” refers to a bicycloalkyl structure in which at least one carbon atom is replaced with a heteroatom independently selected from oxygen, nitrogen, and sulfur.

The term “heterospiroalkyl” refers to a spiroalkyl structure in which at least one carbon atom is replaced with a heteroatom independently selected from oxygen, nitrogen, and sulfur.

The term “alkenyl” refers to an ethylenically unsaturated hydrocarbon group, straight or branched chain, having 1 or 2 ethylenic bonds, for example vinyl, allyl, 1-butenyl, 2-butenyl, isopropenyl, 2-pentenyl, and the like.

Unless otherwise specified, the term “cycloalkenyl” refers to a cyclic aliphatic 3 to 12 ring structure, optionally substituted with alkyl, hydroxy and halo, having 1 or 2 ethylenic bonds such as methylcyclopropenyl, trifluoromethylcyclopropenyl, cyclopentenyl, cyclohexenyl, 1,4-cyclohexadienyl, and the like.

The term “alkynyl” refers to an unsaturated hydrocarbon group, straight or branched, having at least one acetylenic bond, for example ethynyl, propargyl, and the like.

The term “aryl” refers to an all-carbon monocyclic, bicyclic, or polycyclic groups of 6 to 12 carbon atoms having a completely conjugated pi-electron system, which may be optionally substituted. Examples of aryl include, but are not limited to, phenyl, 4-chlorophenyl, 4-fluorophenyl, 4-bromophenyl, 3-nitrophenyl, 2-methoxyphenyl, 2-methylphenyl, 3-methyphenyl, 4-methylphenyl, 4-ethylphenyl, 2-methyl-3-methoxyphenyl, 2,4-dibromophenyl, 3,5-difluorophenyl, 3,5-dimethylphenyl, 2,4,6-trichlorophenyl, 4-methoxyphenyl, naphthyl, 2-chloronaphthyl, 2,4-dimethoxyphenyl, 4-(trifluoromethyl)phenyl, and 2-iodo-4-methylphenyl.

The terms “heteroaryl” refer to a substituted or unsubstituted monocyclic, bicyclic, or polycyclic group of 5 to 12 ring atoms containing one or more ring heteroatoms selected from N, O, and S, the remaining ring atoms being C, and, in addition, having a completely conjugated pi-electron system. Examples of such heteroaryl rings include, but are not limited to, furyl, thienyl, pyrrolyl, pyrazolyl, imidazolyl, oxazolyl, isoxazolyl, thiazolyl, isothiazolyl, triazolyl, oxadiazolyl, thiadiazolyl, tetrazolyl, pyridyl, pyridazinyl, pyrimidinyl, pyrazinyl, and triazinyl. The terms “heteroaryl” also include heteroaryl rings with fused carbocyclic ring systems that are partially or fully unsaturated, such as a benzene ring, to form a benzofused heteroaryl. For example, benzimidazole, benzoxazole, benzothiazole, benzofuran, quinoline, isoquinoline, quinoxaline, and the like. Furthermore, the terms “heteroaryl” include fused 5-6, 5-5, 6-6 ring systems, optionally possessing one nitrogen atom at a ring junction. Examples of such hetaryl rings include, but are not limited to, pyrrolopyrimidinyl, imidazo[1,2-a]pyridinyl, imidazo[2,1-b]thiazolyl, imidazo[4,5-b]pyridine, pyrrolo[2,1-f][1,2,4]triazinyl, and the like. Heteroaryl groups may be attached to other groups through their carbon atoms or the heteroatom(s), if applicable. For example, pyrrole may be connected at the nitrogen atom or at any of the carbon atoms.

The term “heterocycloalkyl” refers to a substituted or unsubstituted monocyclic, bicyclic, or polycyclic ring group having in the ring(s) of 3 to 12 ring atoms, in which one or more ring atoms are heteroatoms selected from N, O, and S, the remaining ring atoms being C. The rings may also have one or more double bonds. However, the rings do not have a completely conjugated pi-electron system. Examples of heterocycloalkyl rings include azetidine, oxetane, tetrahydrofuran, tetrahydropyran, oxepane, oxocane, thietane, thiazolidine, oxazolidine, oxazetidine, pyrazolidine, isoxazolidine, isothiazolidine, tetrahydrothiophene, tetrahydrothiopyran, thiepane, thiocane, azetidine, pyrrolidine, piperidine, N-methylpiperidine, azepane, 1,4-diazapane, azocane, [1,3]dioxane, oxazolidine, piperazine, homopiperazine, morpholine, thiomorpholine, 1,2,3,6-tetrahydropyridine and the like. Other examples of heterocycloalkyl rings include the oxidized forms of the sulfur-containing rings. Thus, tetrahydrothiophene-1-oxide, tetrahydrothiophene-1,1-dioxide, thiomorpholine-1-oxide, thiomorpholine-1,1-dioxide, tetrahydrothiopyran-1-oxide, tetrahydrothiopyran-1,1-dioxide, thiazolidine-1-oxide, and thiazolidine-1,1-dioxide are also considered to be heterocycloalkyl rings. The term “heterocycloalkyl” also includes fused ring systems and can include a carbocyclic ring that is partially or fully unsaturated, such as a benzene ring, to form benzofused heterocycloalkyl rings. For example, 3,4-dihydro-1,4-benzodioxine, tetrahydroquinoline, tetrahydroisoquinoline and the like. The term “heterocycloalkyl” also includes heterobicycloalkyl, heteropolycycloalkyl, or heterospiroalkyl, which are bicycloalkyl, polycycloalkyl, or spiroalkyl, in which one or more carbon atom(s) are replaced by one or more heteroatoms selected from O, N, and S. For example, 2-oxa-spiro[3.3]heptane, 2,7-diaza-spiro[4.5]decane, 6-oxa-2-thia-spiro[3.4]octane, octahydropyrrolo[1,2-a]pyrazine, 7-aza-bicyclo[2.2.1]heptane, 2-oxa-bicyclo[2.2.2]octane, and the like, are such heterocycloalkyls.

The convention “_{x-y}” indicates a moiety containing from x to y atoms, e.g., ₅₋₆heterocycloalkyl means a heterocycloalkyl having five or six ring members.

The term “alkoxy” includes both branched and straight chain terminal alkyl groups attached to a bridging oxygen atom. Typical alkoxy groups include methoxy, ethoxy, n-propoxy, isopropoxy, tert-butoxy and the like.

One in the art understands that an “oxo” requires a second bond from the atom to which the oxo is attached. Accordingly, it is understood that oxo cannot be subststituted onto an aryl or heteroaryl ring.

The term “halo” refers to fluoro, chloro, bromo, or iodo.

The term “pharmaceutically acceptable salts” refers to salts prepared from pharmaceutically acceptable non-toxic bases or acids. When the compound of the present invention is acidic, its corresponding salt can be conveniently prepared from pharmaceutically acceptable non-toxic bases, including inorganic bases and organic bases. Salts derived from such inorganic bases include aluminum, ammonium, calcium, copper (ic and ous), ferric, ferrous, lithium, magnesium, manganese (ic and ous), potassium, sodium, zinc and the like salts. Particularly preferred are the ammonium, calcium, magnesium, potassium and sodium slats. Salts derived from pharmaceutically acceptable organic non-toxic bases include salts of primary, secondary, and tertiary amines, as well as cyclic amines and substituted amines such as naturally occurring and synthesized substituted amines. Other pharmaceutically acceptable organic non-toxic bases from which salts can be formed include ion exchange resins such as, for example, arginine, betaine, caffeine, choline, N′,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like.

When the compound of the present invention is basic, its corresponding salt can be conveniently prepared from pharmaceutically acceptable non-toxic acids, including inorganic and organic acids. Such acids include, for example, acetic, benzenesulfonic, benzoic, camphorsulfonic, citric, ethanesulfonic, formic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid and the like. Preferred are citric, hydrobromic, formic, hydrochloric, maleic, phosphoric, sulfuric and tartaric acids. Particularly preferred are formic and hydrochloric acid.

1. A compound of Formula I: 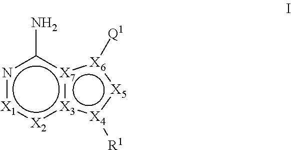
wherein X₁, and X₂ are each independently N or CE¹
X₃, X₄, X₆, and X, are each independently N or C;
X₅ is N, >CH, >CD, or >NE¹;
wherein at least one of X₃, X₄, X₅, X₆, and X, is N or >NE¹;
Q¹ is 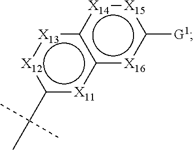
wherein X₁₁, X₁₂, X₁₃, X₁₄, X₁₅, and X₁₆ are each independently N, >C-E¹¹, or >N⁺—O⁻; and at least one of X₁₁, X₁₂, X₁₃, X₁₄, X₁₅, and X₁₆ is N or >N⁺—O⁻;
E¹ is H, D, halo, —CF₃, —OCF₃, —OR², —NR²R³(R^{2a})ⱼ₁, —C(═O)R², —CO₂R², —CONR²R³, —NO₂, —CN, —S(O)ⱼ₁R², —SO₂NR²R³, —NR²C(═O)R³, —NR²C(═O)OR³, —NR²C(═O)NR³R^{2a}, —NR²S(O)ⱼ₁R³, —C(═S)OR², —C(═O)SR², —NR²C(═NR³)NR^{2a}R^{3a}, —NR²C(═NR³)OR^{2a}, —NR²C(═NR³)SR^{2a}, —OC(═O)OR², —OC(═O)NR²R³, —OC(═O)SR², —SC(═O)OR², —SC(═O)NR²R³, C₀₋₁₀alkyl, C₂₋₁₀alkenyl, C₂₋₁₀alkynyl, C₁₋₁₀alkoxyC₁₋₁₀alkyl, C₁₋₁₀alkoxyC₂₋₁₀alkenyl, C₁₋₁₀alkoxyC₂₋₁₀alkynyl, C₁₋₁₀alkylthioC₁₋₁₀alkyl, C₁₋₁₀alkylthioC₂₋₁₀alkenyl, C₁₋₁₀alkylthioC₂₋₁₀alkynyl, cycloC₃₋₈alkyl, cycloC₃₋₈alkenyl, cycloC₃₋₈alkylC₁₋₁₀alkyl, cycloC₃₋₈alkenylC₁₋₁₀alkyl, cycloC₃₋₈alkylC₂₋₁₀alkenyl, cycloC₃₋₈alkenylC₂₋₁₀alkenyl, cycloC₃₋₈alkylC₂₋₁₀alkynyl, cycloC₃₋₈alkenylC₂₋₁₀alkynyl, heterocyclyl-C₀₋₁₀alkyl, heterocyclyl-C₂₋₁₀alkenyl, or heterocyclyl-C₂₋₁₀alkynyl, any of which is optionally substituted with one or more independent D, halo, oxo, —CF₃, —OCF₃, —OR²²², —NR²²²R³³³(R^{222a})—C(═O)R²²², —CO₂R²²², —C(═O)NR²²²R³³³, —NO₂, —CN, —S(═O)ⱼ₁ₐR²²², —SO₂NR²²²R³³³, —NR²²²C(═O)R³³³, —NR²²²C(═O)OR³³³, —NR²²²C(═O)NR³³³R^{222a}, —NR²²²S(O)ⱼ₁ₐR³³³, —C(═S)OR²²², —C(═O)SR²²², —NR²²²C(═NR³³³)NR^{222a}R^{333a}, —NR²²²C(═NR³³³)OR^{222a}, —NR²²²C(═NR³³³)SR^{222a}, —OC(═O)OR²²², —OC(═O)NR²²²R³³³, —OC(═O)SR²²², —SC(═O)OR²²², or —SC(═O)NR²²²R³³³ substituents;
or E¹ is aryl-C₀₋₁₀alkyl, aryl-C₂₋₁₀alkenyl, aryl-C₂₋₁₀alkynyl, hetaryl-C₀₋₁₀alkyl, hetaryl-C₂₋₁₀alkenyl, or hetaryl-C₂₋₁₀alkynyl, any of which is optionally substituted with one or more independent D, halo, —CF₃, —OCF₃, —OR²²², —NR²²²R³³³(R^{222a})ⱼ₂ₐ), —C(O)R²²², —CO₂R²²², —C(═O)NR²²²R³³³, —NO₂, —CN, —S(O)₉ₐR²²², —SO₂NR²²²R³³³, —NR²²²C(═O)R³³³, —NR²²²C(═O)OR³³³, —NR²²²C(═O)NR³³³R^{222a}, —NR²²²S(O)₂ⱼₐR³³³, —C(═S)OR²²², —C(═O)SR²²², —NR²²²C(═NR³³³)NR^{222a}R^{333a}, —NR²²²C(═NR³³³)OR^{222a}, —NR²²²C(═NR³³³)SR^{222a}, —OC(═O)OR²²², —OC(═O)NR²²²R³³³, —OC(═O)SR²²², —SC(═O)OR²²², or —SC(═O)NR²²²R³³³ substituents;
each E¹¹ is independently H, D, halo, —CF₃, —OCF₃, methyl, or ethyl;
G¹ is phenyl or pyridyl, either optionally substituted by one or more D or halogen atoms;
R¹ is absent, D, cycloC₃₋₁₀alkyl, bicycloC₅₋₁₀alkyl, aryl, heteroaryl, aralkyl, heteroaralkyl, heterocyclyl, heterobicycloC₅₋₁₀alkyl, spiroalkyl, or heterospiroalkyl, any of which is optionally substituted by one or more independent G¹¹ substituents;
each G¹¹ is H, D, halo, oxo, —CF₃, —OCF₃, —OR²¹, —NR²¹R³¹(R^{2a1})ⱼ₄), —C(O)R²¹, —CO₂R²¹, —C(═O)NR²¹R³¹, —NO₂, —CN, —S(O)ⱼ₄R²¹, —SO₂NR²¹R³¹, NR²¹(C═O)R³¹, NR²¹C(═O)OR³¹, NR²¹C(═O)NR³¹R^{2a1}, NR²¹S(O)ⱼ₄R³¹, —C(═S)OR²¹, —C(═O)SR²¹, NR²¹C(═NR³¹)NR^{2a1}R^{3a1}, —NR²¹C(═NR³¹)OR^{2a1}, —NR²¹C(═NR³¹)SR^{2a1}, —OC(═O)OR²¹, —OC(═O)NR²¹R³¹, —OC(═O)SR²¹, —SC(═O)OR²¹, —SC(═O)NR²¹R³¹, —P(O)OR²¹OR³¹, C₀₋₁₀alkyl, C₂₋₁₀alkenyl, C₂₋₁₀alkynyl, C₁₋₁₀alkoxyC₁₋₁₀alkyl, C₁₋₁₀alkoxyC₂₋₁₀alkenyl, C₁₋₁₀alkoxyC₂₋₁₀alkynyl, C₁₋₁₀alkylthioC₁₋₁₀alkyl, C₁₋₁₀alkylthioC₂₋₁₀alkenyl, C₁₋₁₀alkylthioC₂₋₁₀alkynyl, cycloC₃₋₈alkyl, cycloC₃₋₈alkenyl, cycloC₃₋₈alkylC₁₋₁₀alkyl, cycloC₃₋₈alkenylC₁₋₁₀alkyl, cycloC₃₋₈alkylC₂₋₁₀alkenyl, cycloC₃₋₈alkenylC₂₋₁₀alkenyl, cycloC₃₋₈alkylC₂₋₁₀alkynyl, cycloC₃₋₈alkenylC₂₋₁₀alkynyl, heterocyclyl-C₀₋₁₀alkyl, heterocyclyl-C₂₋₁₀alkenyl, or heterocyclyl-C₂₋₁₀alkynyl,
any of which is optionally substituted with one or more independent D, halo, oxo, —CF₃, —OCF₃, —OR²²²¹, —NR²²²¹R³³³¹(R^{222a1})ⱼ₄ₐ, —C(O)R²²²¹, —CO₂R²²²¹, —C(═O)NR²²²¹R³³³¹, —NO₂, —CN, —S(O)ⱼ₄ₐR²²²¹, —SO₂NR²²²¹R³³³¹, —NR²²²¹C(═O)R³³³¹, —NR²²²¹C(═O)OR³³³¹, —NR²²²¹C(═O)NR³³³¹R^{222a1}, —NR²²²¹S(O)ⱼ₄ₐR³³³¹, —C(═S)OR²²²¹, —C(═O)SR²²²¹, —NR²²²¹C(═NR³³³¹)NR^{222a1}R^{333a1}, —NR²²²¹C(═NR³³³¹)OR^{222a1}, —NR²²²¹C(═NR³³³¹)SR^{222a1}, —C(═O)OR²²²¹, —OC(═O)NR²²²¹R³³³¹, —OC(═O)SR²²²¹, —SC(═O)OR²²²¹, —P(O)OR²²²¹OR³³³¹, or —SC(═O)NR²²²¹R³³³¹ substituents;
or G¹¹ is aryl-C₀₋₁₀alkyl, aryl-C₂₋₁₀alkenyl, aryl-C₂₋₁₀alkynyl, hetaryl-C₀₋₁₀alkyl, hetaryl—C₂₋₁₀alkenyl, or hetaryl-C₂₋₁₀alkynyl, any of which is optionally substituted with one or more independent D, halo, —CF₃, —OCF₃, —OR²²²¹, —NR²²²¹R³³³¹ (R^{222a1})ⱼ₅ₐ, —C(O)R²²²¹, —CO₂R²²²¹, —C(═O)NR²²²¹R³³³¹, —NO₂, —CN, —S(O)ⱼ₅ₐR²²²¹, —SO₂NR²²²¹R³³³¹, —NR²²²¹C(═O)R³³³¹, —NR²²²¹C(═O)OR³³³¹, —NR²²²¹C(═O)NR³³³¹R^{222a1}, —NR²²²¹S(O)ⱼ₅ₐR³³³¹, —C(═S)OR²²²¹, —C(═O)SR²²²¹, —NR²²²¹C(═NR³³³¹)NR^{222a1}R^{333a1}, —NR²²²¹ C(═NR³³³¹)OR^{222a1}, —NR²²²¹ C(═NR³³³¹)SR^{222a1}, —OC(═O)OR²²²¹, —OC(═O)NR²²²¹R³³³¹, —OC(═O)SR²²²¹, —SC(═O)OR²²²¹, —P(O)OR²²²¹OR³³³¹, or —SC(═O)NR²²²¹R³³³¹ substituents;
or G¹¹ is C, taken together with the carbon to which it is attached forms a C═C double bond which is substituted with R⁵ and G¹″;
R², R^{2a}, R³, R^{3a}, R²²², R^{222a}, R³³³, R^{333a}, R²¹, R^{2a1}, R³¹, R^{3a1}, R²²²¹, R^{222a1}, R³³³¹, and R^{333a1} are each independently C₀₋₁₀alkyl, C₂₋₁₀alkenyl, C₂₋₁₀alkynyl, C₁₋₁₀alkoxyC₂₋₁₀alkenyl, C₁₋₁₀alkoxyC₂₋₁₀alkynyl, C₁₋₁₀alkylthioC₂₋₁₀alkenyl, C₁₋₁₀alkylthioC₂₋₁₀alkynyl, cycloC₃₋₈alkyl, cycloC₃₋₈alkenyl, cycloC₃₋₈alkylC₁₋₁₀alkyl, cycloC₃₋₈alkenylC₁₋₁₀alkyl, cycloC₃₋₈alkylC₂₋₁₀alkenyl, cycloC₃₋₈alkenylC₂₋₁₀alkenyl, cycloC₃₋₈alkylC₂₋₁₀alkynyl, cycloC₃₋₈alkenylC₂₋₁₀alkynyl, heterocyclyl-C₀₋₁₀alkyl, heterocyclyl-C₂₋₁₀alkenyl, heterocyclyl-C₂₋₁₀alkynyl, aryl-C₀₋₁₀alkyl, aryl-C₂₋₁₀alkenyl, or aryl-C₂₋₁₀alkynyl, hetaryl-C₀₋₁₀alkyl, hetaryl-C₂₋₁₀alkenyl, or hetaryl-C₂₋₁₀alkynyl, any of which is optionally substituted by one or more independent G¹¹¹ substituents; or in the case of —NR²R³(R^{2a})ⱼ₁ or —NR²²²R³³³(R^{222a})ⱼ₁ₐ—NR²²²R³³³(R^{222a})ⱼ₂ₐ or —NR²¹R³¹(R^{2a1})ⱼ₄ or —NR²²²¹R³³³¹(R^{222a1})ⱼ₄ₐ or —NR²²²¹R³³³¹(R^{222a1})ⱼ₅ₐ, then R² and R³, or R²²² and R³³³, or R²²²¹ and R³³³¹, respectively, are optionally taken together with the nitrogen atom to which they are attached to form a 3-10 membered saturated or unsaturated ring, wherein said ring is optionally substituted by one or more independent G¹¹¹¹ substituents and wherein said ring optionally includes one or more heteroatoms other than the nitrogen to which R² and R³, or R²²² and R³³³, or R²²²¹ and R³³³¹ are attached;
R⁵, G¹¹¹, and G¹¹¹¹ are each independently C₀₋₁₀alkyl, C₂₋₁₀alkenyl, C₂₋₁₀alkynyl, C₁₋₁₀alkoxyC₁₋₁₀alkyl, C₁₋₁₀alkoxyC₂₋₁₀alkenyl, C₁₋₁₀alkoxyC₂₋₁₀alkynyl, C₁₋₁₀alkylthioC₁₋₁₀alkyl, C₁₋₁₀alkylthioC₂₋₁₀alkenyl, C₁₋₁₀alkylthioC₂₋₁₀alkynyl, cycloC₃₋₈alkyl, cycloC₃₋₈alkenyl, cycloC₃₋₈alkylC₁₋₁₀alkyl, cycloC₃₋₈alkenylC₁₋₁₀alkyl, cycloC₃₋₈alkylC₂₋₁₀alkenyl, cycloC₃₋₈alkenylC₂₋₁₀alkenyl, cycloC₃₋₈alkylC₂₋₁₀alkynyl, cycloC₃₋₈alkenylC₂₋₁₀alkynyl, heterocyclyl-C₀₋₁₀alkyl, heterocyclyl-C₂₋₁₀alkenyl, heterocyclyl-C₂₋₁₀alkynyl, aryl-C₂₋₁₀alkenyl, aryl-C₂₋₁₀alkynyl, hetaryl-C₀₋₁₀alkyl, hetaryl-C₂₋₁₀alkenyl, or hetaryl-C₂₋₁₀alkynyl, any of which is optionally substituted with one or more independent D, halo, —CF₃, —OCF₃, —OR⁷⁷, —NR⁷⁷R⁸⁷, —C(O)R⁷⁷, —CO₂R⁷⁷, —CONR⁷⁷R⁸⁷, —NO₂, —CN, —S(O)ⱼ₅ₐR⁷⁷, —SO₂NR⁷⁷R⁸⁷, —NR⁷⁷C(═O)R⁸⁷, —NR⁷⁷C(═O)OR⁸⁷, —NR⁷⁷C(═O)NR⁷⁸R⁸⁷, —NR⁷⁷S(O)ⱼ₅ₐR⁸⁷, —C(═S)OR⁷⁷, —C(═O)SR⁷⁷, —NR⁷⁷C(═NR⁸⁷)NR⁷⁸R⁸⁸, —NR⁷⁷C(═NR⁸⁷)OR⁷⁸, —NR⁷⁷C(═NR⁸⁷)SR⁷⁸, —OC(═O)OR⁷⁷, —OC(═O)NR⁷⁷R⁸⁷, —OC(═O)SR⁷⁷, —SC(═O)OR⁷⁷, —P(O)OR⁷⁷OR⁸⁷, or —SC(═O)NR⁷⁷R⁸⁷ substituents;
R⁷⁷, R⁷⁸, R⁸⁷, and R⁸⁸ are each independently C₀₋₁₀alkyl, C₂₋₁₀alkenyl, C₂₋₁₀alkynyl, C₁₋₁₀alkoxyC₁₋₁₀alkyl, C₁₋₁₀alkoxyC₂₋₁₀alkenyl, C₁₋₁₀alkoxyC₂₋₁₀alkynyl, C₁₋₁₀alkylthioC₁₋₁₀alkyl, C₁₋₁₀alkylthioC₂₋₁₀alkenyl, C₁₋₁₀alkylthioC₂₋₁₀alkynyl, cycloC₃₋₈alkyl, cycloC₃₋₈alkenyl, cycloC₃₋₈alkylC₁₋₁₀alkyl, cycloC₃₋₈alkenylC₁₋₁₀alkyl, cycloC₃₋₈alkylC₂₋₁₀alkenyl, cycloC₃₋₈alkenylC₂₋₁₀alkenyl, cycloC₃₋₈alkylC₂₋₁₀alkynyl, cycloC₃₋₈alkenylC₂₋₁₀alkynyl, heterocyclyl-C₀₋₁₀alkyl, heterocyclyl-C₂₋₁₀alkenyl, heterocyclyl-C₂₋₁₀alkynyl, C₁₋₁₀alkylcarbonyl, C₂₋₁₀alkenylcarbonyl, C₂₋₁₀alkynylcarbonyl, C₁₋₁₀alkoxycarbonyl, C₁₋₁₀alkoxycarbonylC₁₋₁₀alkyl, monoC₁₋₆alkylaminocarbonyl, diC₁₋₆alkylaminocarbonyl, mono(aryl)aminocarbonyl, di(aryl)aminocarbonyl, or C₁₋₁₀alkyl(aryl)aminocarbonyl, any of which is optionally substituted with one or more independent halo, cyano, hydroxy, nitro, C₁₋₁₀alkoxy, —SO₂N(C₀₋₄alkyl)(C₀₋₄alkyl), or —N(C₀₋₄alkyl)(C₀₋₄alkyl) substituents;
or R⁷⁷, R⁷⁸, R⁸⁷, and R⁸⁸ are each independently aryl-C₀₋₁₀alkyl, aryl-C₂₋₁₀alkenyl, aryl-C₂₋₁₀alkynyl, hetaryl-C₀₋₁₀alkyl, hetaryl-C₂₋₁₀alkenyl, hetaryl-C₂₋₁₀alkynyl, mono(C₁₋₆alkyl)aminoC₁₋₆alkyl, di(C₁₋₆alkyl)aminoC₁₋₆alkyl, mono(aryl)aminoC₁₋₆alkyl, di(aryl)aminoC₁₋₆alkyl, or —N(C₁₋₆alkyl)-C₁₋₆alkyl-aryl, any of which is optionally substituted with one or more independent halo, cyano, nitro, —O(C₀₋₄alkyl), C₂₋₁₀alkenyl, C₂₋₁₀alkynyl, haloC₂₋₁₀alkenyl, haloC₂₋₁₀alkynyl, —COOH, C₁₋₄alkoxycarbonyl, —CON(C₀₋₄alkyl)(C₀₋₁₀alkyl), —SO₂N(C₀₋₄alkyl)(C₀₋₄alkyl), or —N(C₀₋₄alkyl)(C₀₋₄alkyl) substituents;
j1, j1a, j2a, j4, j4a, and j5a are each independently 0, 1, or 2;
or a pharmaceutically acceptable salt thereof, wherein any hydrogen atom can be replaced by a D atom and the compound or salt is present as a material comprising at least one D atom in an abundance of at least about 10%.

2. The compound or salt of claim 1, having the formula: 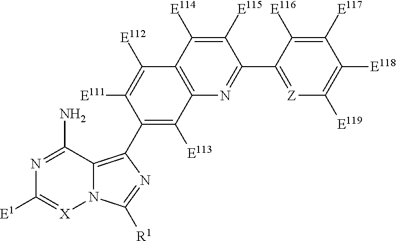
wherein:
X is N, >CH, or >CD;
Z is N, >CH, >CD, or >C-halogen;
R¹ is phenyl, cycloC₃₋₆alkyl, bicycloC₆₋₁₀alkyl, spiroalkyl, or heteroalkyl, any of which is optionally substituted by one or more G¹¹ substituents;
E¹ is H or D;
each E¹¹¹-E¹¹⁵ is independently H, D, halogen, —CF₃, methyl, or ethyl;
each E¹¹⁶-E¹¹⁹ is independently H, D, or halogen;
which is present as a material comprising at least one D atom in an abundance of at least about 20%.

3. The compound or salt of claim 2, wherein R¹ is cycloC₃₋₆alkyl optionally substituted by one or more G¹¹ substituents.

4. The compound or salt of claim 1, having the formula: 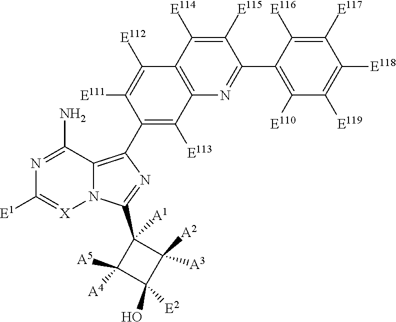
wherein:
X is N, >CH, or >CD;
E¹ is H or D;
each E¹¹¹-E¹¹⁵ is independently H, D, halo, —CF₃, or methyl;
each E¹¹⁰ and E¹¹⁶-E¹¹⁹ is independently H, D, or halogen;
each A¹-A⁵ is independently H or D,
E² is —CH₃, CH₂D, CHD₂, or CD₃;
which is present as a material comprising at least one said D atom in an abundance of at least about 30%.

5. The compound or salt of claim 4, wherein:
X is CH or CD;
each of A¹-A⁵, E¹, E¹¹⁰-E¹¹⁹ is independently H or D;
E² is —CH₃, CH₂D, CHD₂, or CD₃;
which is present as a material comprising at least one said D atom in an abundance of at least about 40%.

6. The compound or salt of claim 5, which is present as a material comprising at least 1 to 3 said D atoms each in an abundance of at least about 50%.

7. The compound of any of the examples herein, which is present as a material comprising the incorporated D atom(s) each in an abundance of at least about 50%.

8. The compound or salt of claim 5, which is present as a material in which each atom designated as deuterium has a deuterium abundance of at least about 50%.

9. The compound or salt of claim 5, which is present as a material in which at least one atom designated as deuterium has a deuterium abundance of at least about 90%.

10. The compound or salt of claim 9, which is present as a material in which each atom not designated as deuterium has substantially its natural isotopic abundance.

11. The compound or salt of claim 10, which inhibits IGF-1R with an IC₅₀ of about 1 μM or less in a cellular assay.

12. The compound or salt of claim 11, which is present as a material that is substantially stereochemically pure.

13. A pharmaceutical composition comprising the compound or salt of claim 12, formulated with or without one or more pharmaceutical carriers.

14.-20. (canceled)
